SEQUENCE LISTING (16 sequences)

SEQ ID NO: 1 - DNA, 29 bp, Artificial Sequence
  Description of Artificial Sequence PCR primer for amplifying HES1 promoter from mouse genomic DNA
ggggtaccctcaggcgcgcgccattggcc

SEQ ID NO: 2 - DNA, 29 bp, Artificial Sequence
  Description of Artificial Sequence PCR primer for amplifying HES1 promoter from mouse genomic DNA
gaagatctgcttacgtccttttacttgac

SEQ ID NO: 3 - DNA, 26 bp, Artificial Sequence
  Description of Artificial Sequence Adenovirus major late promoter TATA-box motif with BglII and HindIII cohesive ends
gatctggggggctataaaagggggta

SEQ ID NO: 4 - DNA, 26 bp, Artificial Sequence
  Description of Artificial Sequence Adenovirus major late promoter TATA-box motif with BglII and HindIII cohesive ends
agcttacccccttttatagcccccca

SEQ ID NO: 5 - DNA, 61 bp, Artificial Sequence
  Description of Artificial Sequence CBF-1 promoter tetramer with XhoI and BglII cohesive ends
tcgagaccgtgggaacttaaccgtgggaacttaaccgtgggaacttaaccgtgggaactta

SEQ ID NO: 6 - DNA, 61 bp, Artificial Sequence
  Description of Artificial Sequence CBF-1 promoter tetramer with XhoI and BglII cohesive ends
gatctaagttcccacggttaagttcccacggttaagttcccacggttaagttcccacggtc

SEQ ID NO: 7 - DNA, 39 bp, Artificial Sequence
  Description of Artificial Sequence PCR amplimer for generating a truncated fragment of human Notch1 cDNA
aaaggatccaccatggcacgcaagcgccggcgcagtcat

SEQ ID NO: 8 - DNA, 31 bp, Artificial Sequence
  Description of Artificial Sequence PCR amplimer for generating a truncated fragment of human Notch1 cDNA
gcgcctcgagttagtccacgggcgagagcat

SEQ ID NO: 9 - DNA, 91 bp, Artificial Sequence
  Description of Artificial Sequence Oligo annealed to the NIC2202 sequence to remove the stop codon from the NIC2202 fragment of human Notch1 cDNA
cctggcctgtggaagcaaggaggccaaggacctcaaggcacggaggaagaagtcccaggatggcaagggctgcctgctggacggcggccgc

SEQ ID NO: 10 - DNA, 95 bp, Artificial Sequence
  Description of Artificial Sequence Oligo annealed to the NIC2202 sequence to remove the stop codon from the NIC2202 fragment of human Notch1 cDNA
tcgagcggccgccgtccagcaggcagcccttgccatcctgggacttcttcctccgtgccttgaggtccttggcctccttgcttccacaggccagg

SEQ ID NO: 11 - protein, 2556 aa, Homo sapiens
  MOD_RES (891): Variable amino acid
MPPLLAPLLCLALLPALAARGPRCSQPGETCLNGGKCEAANGTEACVCGGAFVGPRCQDPNPCLSTPCKNAGTCHVVDRRGVADYACSCALGFSGPLCLTPLDNACLTNPCRNGGTCDLLTLTEYKCRCPPGWSGKSCQQADPCASNPCANGGQCLPFEASYICHCPPSFHGPTCRQDVNECGQKPRLCRHGGTCHNEVGSYRCVCRATHTGPNCERPYVPCSPSPCQNGGTCRPTGDVTHECACLPGFTGQNCEENIDDCPGNNCKNGGACVDGVNTYNCPCPPEWTGQYCTEDVDECQLMPNACQNGGTCHNTHGGYNCVCVNGWTGEDCSENIDDCASAACFHGATCHDRVASFYCECPHGRTGLLCHLNDACISNPCNEGSNCDTNPVNGKAICTCPSGYTGPACSQDVDECSLGANPCEHAGKCINTLGSFECQCLQGYTGPRCEIDVNECVSNPCQNDATCLDQIGEFQCMCMPGYEGVHCEVNTDECASSPCLHNGRCLDKINEFQCECPTGFTGHLCQYDVDECASTPCKNGAKCLDGPNTYTCVCTEGYTGTHCEVDIDECDPDPCHYGSCKDGVATFTCLCRPGYTGHHCETNINECSSQPCRLRGTCQDPDNAYLCFCLKGTTGPNCEINLDDCASSPCDSGTCLDKIDGYECACEPGYTGSMCNSNIDECAGNPCHNGGTCEDGINGFTCRCPEGYHDPTCLSEVNECNSNPCVHGACRDSLNGYKCDCDPGWSGTNCDINNNECESNPCVNGGTCKDMTSGIVCTCREGFSGPNCQTNINECASNPCLNKGTCIDDVAGYKCNCLLPYTGATCEVVLAPCAPSPCRNGGECRQSEDYESFSCVCPTAGAKGQTCEVDINECVLSPCRHGASCQNTHGXYRCHCQAGYSGRNCETDIDDCRPNPCHNGGSCTDGINTAFCDCLPGFRGTFCEEDINECASDPCRNGANCTDCVDSYTCTCPAGFSGIHCENNTPDCTESSCFNGGTCVDGINSFTCLCPPGFTGSYCQHVVNECDSRPCLLGGTCQDGRGLHRCTCPQGYTGPNCQNLVHWCDSSPCKNGGKCWQTHTQYRCECPSGWTGLYCDVPSVSCEVAAQRQGVDVARLCQHGGLCVDAGNTHHCRCQAGYTGSYCEDLVDECSPSPCQNGATCTDYLGGYSCKCVAGYHGVNCSEEIDECLSHPCQNGGTCLDLPNTYKCSCPRGTQGVHCEINVDDCNPPVDPVSRSPKCFNNGTCVDQVGGYSCTCPPGFVGERCEGDVNECLSNPCDARGTQNCVQRVNDFHCECRAGHTGRRCESVINGCKGKPCKNGGTCAVASNTARGFICKCPAGFEGATCENDARTCGSLRCLNGGTCISGPRSPTCLCLGPFTGPECQFPASSPCLGGNPCYNQGTCEPTSESPFYRCLCPAKFNGLLCHILDYSFGGGAGRDIPPPLIEEACELPECQEDAGNKVCSLQCNNHACGWDGGDCSLNFNDPWKNCTQSLQCWKYFSDGHCDSQCNSAGCLFDGFDCQRAEGQCNPLYDQYCKDHFSDGHCDQGCNSAECEWDGLDCAEHVPERLAAGTLVVVVLMPPEQLRNSSFHFLRELSRVLHTNVVFKRDAHGQQMIFPYYGREEELRKHPIKRAAEGWAAPDALLGQVKASLLPGGSEGGRRRRELDPMDVRGSIVYLEIDNRQCVQASSQCFQSATDVAAFLGALASLGSLNIPYKIEAVQSETVEPPPPAQLHFMYVAAAAFVLLFFVGCGVLLSRKRRRQHGQLWFPEGFKVSEASKKKRREPLGEDSVGLKPLKNASDGALMDDNQNEWGDEDLETKKFRFEEPVVLPDLDDQTDHRQWTQQHLDAADLRMSAMAPTPPQGEVDADCMDVNVRGPDGFTPLMIASCSGGGLETGNSEEEEDAPAVISDFIYQGASLHNQTDRTGETALHLAARYSRSDAAKRLLEASADANIQDNMGRTPLHAAVSADAQGVFQILIRNRATDLDARMHDGTTPLILAARLAVEGMLEDLINSHADVNAVDDLGKSALHWAAAVNNVDAAVVLLKNGANKDMQNNREETPLFLAAREGSYETAKVLLDHFANRDITDHMDRLPRDIAQERMHHDIVRLLDEYNLVRSPQLHGAPLGGTPTLSPPLCSPNGYLGSLKPGVQGKKVRKPSSKGLACGSKEAKDLKARRKKSQDGKGCLLDSSGMLSPVDSLESPHGYLSDVASPPLLPSPFQQSPSVPLNHLPGMPDTHLGIGHLNVAAKPEMAALGGGGRLAFETGPPRLSHLPVASGTSTVLGSSSGGALNFTVGGSTSLNGQCEWLSRLQSGMVPNQYNPLRGSVAPGPLSTQAPSLQHGMVGPLHSSLAASALSQMMSYQGLPSTRLATQPHLVQTQQVQPQNLQMQQQNLQPANIQQQQSLQPPPPPPQPHLGVSSAASGHLGRSFLSGEPSQADVQPLGPSSLAVHTILPQESPALPTSLPSSLVPPVTAAQFLTPPSQHSYSSPVDNTPSHQLQVPEHPFLTPSPESPDQWSSSSPHSNVSDWSEGVSSPPTSMQSQIARIPEAFK

SEQ ID NO: 12 - protein, 2471 aa, Homo sapiens
MPALRPALLWALLALWLCCAAPAHALQCRDGYEPCVNEGMCVTYHNGTGYCKCPEGFLGEYCQHRDPCEKNRCQNGGTCVAQAMLGKATCRCASGFTGEDCQYSTSHPCFVSRPCLNGGTCHMLSRDTYECTCQVGFTGKECQWTDACLSHPCANGSTCTTVANQFSCKCLTGFTGQKCETDVNECDIPGHCQHGGTCLNLPGSYQCQCPQGFTGQYCDSLYVPCAPSPCVNGGTCRQTGDFTFECNCLPGFEGSTCERNIDDCPNHRCQNGGVCVDGVNTYNCRCPPQWTGQFCTEDVDECLLQPNACQNGGTCANRNGGYGCVCVNGWSGDDCSENIDDCAFASCTPGSTCIDRVASFSCMCPEGKAGLLCHLDDACISNPCHKGALCDTNPLNGQYICTCPQGYKGADCTEDVDECAMANSNPCEHAGKCVNTDGAFHCECLKGYAGPRCEMDINECHSDPCQNDATCLDKIGGFTCLCMPGFKGVHCELEINECQSNPCVNNGQCVDKVNRFQCLCPPGFTGPVCQIDIDDCSSTPCLNGAKCIDHPNGYECQCATGFTGVLCEENIDNCDPDPCHHGQCQDGIDSYTCICNPGYMGAICSDQIDECYSSPCLNDGRCIDLVNGYQCNCQPGTSGVNCEINFDDCASNPCIHGICMDGINRYSCVCSPGFTGQRCNIDIDECASNPCRKGATCINGVNGFRCICPEGPHHPSCYSQVNECLSNPCIHGNCTGGLSGYKCLCDAGWVGINCEVDKNECLSNPCQNGGTCDNLVNGYRCTCKKGFKGYNCQVNIDECASNPCLNQGTCFDDISGYTCHCVLPYTGKNCQTVLAPCSPNPCENAAVCKESPNFESYTCLCAPGWQGQRCTIDIDECISKPCMNHGLCHNTQGSYMCECPPGFSGMDCEEDIDDCLANPCQNGGSCMDGVNTFSCLCLPGFTGDKCQTDMNECLSEPCKNGGTCSDYVNSYTCKCQAGFDGVHCENNINECTESSCFNGGTCVDGINSFSCLCPVGFTGSFCLHEINECSSHPCLNEGTCVDGLGTYRCSCPLGYTGKNCQTLVNLCSRSPCKNKGTCVQKKAESQCLCPSGWAGAYCDVPNVSCDIAASRRGVLVEHLCQHSGVCINAGNTHYCQCPLGYTGSYCEEQLDECASNPCQHGATCSDFIGGYRCECVPGYQGVNCEYEVDECQNQPCQNGGTCIDLVNHFKCSCPPGTRGLLCEENIDDCARGPHCLNGGQCMDRIGGYSCRCLPGFAGERCEGDINECLSNPCSSEGSLDCIQLTNDYLCVCRSAFTGRHCETFVDVCPQMPCLNGGTCAVASNMPDGFICRCPPGFSGARCQSSCGQVKCRKGEQCVHTASGPRCFCPSPRDCESGCASSPCQHGGSCHPQRQPPYYSCQCAPPFSGSRCELYTAPPSTPPATCLSQYCADKARDGVCDEACNSHACQWDGGDCSLTMENPWANCSSPLPCWDYINNQCDELCNTVECLFDNFECQGNSKTCKYDKYCADHFKDNHCNQGCNSEECGWDGLDCAADQPENLAEGTLVIVVLMPPEQLLQDARSFLRALGTLLHTNLRIKRDSQGELMVYPYYGEKSAAMKKQRMTRRSLPGEQEQEVAGSKVFLEIDNRQCVQDSDHCFKNTDAAAALLASHAIQGTLSYPLVSVVSESLTPERTQLLYLLAVAVVIILFIILLGVIMAKRKRKHGSLWLPEGFTLRRDASNHKRREPVGQDAVGLKNLSVQVSEANLIGTGTSEHWVDDEGPQPKKVKAEDEALLSEEDDPIDRRPWTQQHLEAADIRRTPSLALTPPQAEQEVDVLDVNVRGPDGCTPLMLASLRGGSSDLSDEDEDAEDSSANIITDLVYQGASLQAQTDRTGEMALHLAARYSRADAAKRLLDAGADANAQDNMGRCPLHAAVAADAQGVFQILIRNRVTDLDARMNDGTTPLILAARLAVEGMVAELINCQADVNAVDDHGKSALHWAAAVNNVEATLLLLKNGANRDMQDNKEETPLFLAAREGSYEAAKILLDHFANRDITDHMDRLPRDVARDRMHHDIVRLLDEYNVTPSPPGTVLTSALSPVICGPNRSFLSLKHTPMGKKSRRPSAKSTMPTSLPNLAKEAKDAKGSRRKKSLSEKVQLSESSVTLSPVDSLESPHTYVSDTTSSPMITSPGILQASPNPMLATAAPPAPVHAQHALSFSNLHEMQPLAHGASTVLPSVSQLLSHHHIVSPGSGSAGSLSRLHPVPVPADWMNRMEVNETQYNEMFGMVLAPAEGTHPGIAPQSRPPEGKHITTPREPLPPIVTFQLIPKGSIAQPAGAPQPQSTCPPAVAGPLPTMYQIPEMARLPSVAFPTAMMPQQDGQVAQTILPAYHPFPASVGKYPTPPSQHSYASSNAAERTPSHSGHLQGEHPYLTPSPESPDQWSSSSPHSASDWSDVTTSPTPGGAGGGQRGPGTHMSEPPHNNMQVYA

SEQ ID NO: 13 - protein, 16 aa, Drosophila melanogaster
RQIKIWFQNRRMKWKK

SEQ ID NO: 14 - protein, 16 aa, Artificial Sequence
  Description of Artificial Sequence Illustrative translocating peptide
KWKKKWKKKWKKKWKK

SEQ ID NO: 15 - protein, 12 aa, Artificial Sequence
  Description of Artificial Sequence Illustrative translocating peptide
KWKKKWKKKGGC

SEQ ID NO: 16 - protein, 4 aa, Artificial Sequence
  Description of Artificial Sequence Illustrative translocating peptide fragment
KWKK

(19) United States
(12) Patent Application Publication
(10) Pub. No.: US 2004/0203099 A1
(43) Pub. Date: Oct. 14, 2004

(54) Conjugate of a transport protein and a protein for modulation of notch signalling

(76) Inventors: Roberto Celeste Ercole Solari, Cambridge (GB); Brian Robert Champion, Cambridge (GB); George Albert Ward, Cambridge (GB)

Correspondence Address: FROMMER LAWRENCE & HAUG, 745 FIFTH AVENUE- 10TH FL., NEW YORK, NY, 10151, US

(21) Appl. No.: 10/720,896

(22) Filed: Nov. 24, 2003

Related U.S. Application Data
(63) Continuation-in-part of application No. PCT/GB02/02438, filed on May 24, 2002.

(30) Foreign Application Priority Data
May 25, 2001 (GB) 0112818.0

(51) Int. Cl. C07K 14/705; C07H 21/04

(52) U.S. Cl. 435/69.1; 435/320.1; 435/325; 530/350; 536/23.2

(57) ABSTRACT

Provided is a conjugate comprising a first sequence and a second sequence, wherein the first sequence comprises a transport protein and the second sequence comprises a protein for Notch signalling modulation or a polynucleotide sequence encoding said protein for Notch signalling modulation.

### CROSS-REFERENCE TO RELATED APPLICATIONS

This application is a continuation-in-part of International Application No. PCT/GB02/02438, filed on May 24, 2002, published as WO 02/096952 on Dec. 5, 2002, and claiming priority to GB application Ser. No. 0112818.0, filed on May 25, 2001. Reference is made to U.S. application Ser. No. 09/310,685, filed on May 4, 1999, to U.S. application Ser. No.09/870,902, filed on May 31, 2001, to U.S. application Ser. No. 10/013,310, filed on Dec. 7, 2001, to U.S. application Ser. No. 10/147,354, filed on May 16, 2002, to U.S. application Ser. No. 10/357,321, filed on Feb. 3, 2002, and to U.S. application Ser. No.10/682,230, filed on Oct. 9, 2003.

All of the foregoing applications, as well as all documents cited in the foregoing applications (“application documents”) and all documents cited or referenced in the application documents are incorporated herein by reference. Also, all documents cited in this application (“herein-cited documents”) and all documents cited or referenced in herein-cited documents are incorporated herein by reference. In addition, any manufacturer 's instructions or catalogues for any products cited or mentioned in each of the application documents or herein-cited documents are incorporated by reference. Documents incorporated by reference into this text or any teachings therein can be used in the practice of this invention. Documents incorporated by reference into this text are not admitted to be prior art.

### FIELD OF THE INVENTION

The present invention relates to a molecule and method for targeting of a protein for Notch signalling modulation.

### BACKGROUND OF THE INVENTION

Immunological tolerance to self-antigens is vital to the proper functioning of the mammalian immune system. In addition to the deletion of self-reacting T cells in the thymus, active suppression mediated by regulatory T cells has recently been identified as an important mechanism for maintaining peripheral tolerance (WO98/20142). In autoimmune diseases such as multiple sclerosis, rheumatoid arthritis or diabetes, there is a failure of the proper regulation of tolerance. Improved treatment methods for re-establishing tolerance are desirable for autoimmune diseases. Similarly in allergic conditions and for transplantation of an organ or tissue from a donor individual, induction of tolerance to particular foreign antigens or profiles of foreign antigens is desirable.

It has recently been shown that it is possible to generate a class of regulatory T cells which are able to transmit antigen-specific tolerance to other T cells, a process termed infectious tolerance (WO98/20142). The functional activity of these cells can be mimicked by over-expression of a Notch ligand protein on their cell surfaces or on the surface of antigen presenting cells. In particular, regulatory T cells can be generated by over-expression of a member of the Delta or Serrate family of Notch ligand proteins. Delta or Serrate induced T cells specific to one antigenic epitope are also able to transfer tolerance to T cells recognising, other epitopes on the same or related antigens, a phenomenon termed “epitope spreading”.

In addition, WO00/36089 describes a method for producing a lymphocyte or antigen presenting cell (APC) having or able to induce tolerance to an allergen or antigen which method comprises incubating a lymphocyte or APC obtained from a human or animal patient with (i) a composition capable of upregulating expression of an endogenous Notch or Notch ligand in the lymphocyte and/or APC and (ii) the allergen or antigen.

However, there remains a need in the art for the provision of further diagnostic or therapeutic compositions useful in the detection, prevention and treatment of diseases, such as T cell mediated diseases or disorders. The present invention addresses this problem by delivering an effective Notch signal directly to cells, such as T cells.

### SUMMARY OF THE INVENTION

According to one aspect of the present invention there is provided a conjugate comprising first and second sequences, wherein the first sequence comprises a transport protein or a polynucleotides coding for a transport protein and the second sequence comprises a polypeptide or polynucleotide for Notch signalling modulation.

The present invention relates to a conjugate which is a molecule comprising at least one transport protein linked to at least one polypeptide or polynucleoiide for Notch signalling modulation formed through genetic fusion or chemical coupling. By “linked” we mean that the first and second sequences are associated such that the second sequence is able to be transported by the first sequence into a target cell and/or into a compartment of a target cell. Thus, conjugates include fusion proteins in which the transport protein is linked to a protein for Notch signalling modulation via their polypeptide backbones through genetic expression of a DNA molecule encoding these proteins, directly synthesised proteins and coupled proteins in which pre-formed sequences are associated by a cross-linking agent. The term is also used herein to include associations, such as aggregates, of the protein for Notch signalling modulation with the transport protein. According to one embodiment the second sequence may comprise a,polynucleotide sequence, e.g. a nucleic acid binding domain (such as a transcription factor binding site) or an antisense sequence. This embodiment may be seen as a protein/nucleic acid complex.

The second sequence may be from the same species as the first sequence, but is present in the conjugate of the invention in a manner different from the natural situation, or from a different species.

The conjugates of the present invention are capable of being taken up by a population of cells or directed to a compartment within a cell so that an effector function corresponding to the polypeptide sequence coupled to the transport amino acid sequence can take place within the target cell.

The second sequence of the present invention is a polypeptide or polynucleotide which is for Notch signalling modulation. Notch signalling modulation involves transduction, activation or inhibition of the Notch signalling pathway including upstream and downstream events.

By a polypeptide or polynucleotide which is for Notch signalling transduction we include a molecule which participates in signalling through Notch receptors including activation of Notch, the downstream events of the Notch signalling pathway, transcriptional regulation of downstream target genes and other non-transcriptional downstream events (e.g. post-translational modification of existing proteins). More particularly, the second sequence is a domain that allows activation of target genes of the. Notch signalling pathway, or a polynucleotide sequence which codes therefor.

In other words, by modulating Notch signalling transduction includes:

a) activation of the Notch signalling pathway by (i) dominant negative or inhibitors of repressors and (ii) activators; and

b) blockade of the Notch signalling pathway by (i) dominant negative or inhibitors of activators and (ii) inhibitors.

A very important component of the Notch signalling pathway is Notch receptor/Notch ligand interaction. Notch signalling may involve changes in activity of Notch signalling pathway membrane proteins or G-proteins or Notch signalling pathway enzymes such as proteases, kinases (e.g. serine/threonine kinases), phosphatases, ligases (e.g. ubiquitin ligases) or glycosyltransferases. Alternatively the signalling may involve changes in expression, nature, amount or activity of DNA binding elements such as transcription factors.

In a preferred form of the invention the signalling may be specific signalling, meaning that the signal results substantially or at least predominantly from the Notch signalling pathway, and preferably from Notch/Notch ligand interaction, rather than any other significant interfering or competing cause, such as, for example, cytokine signalling. The Notch signalling pathway is described in more detail below.

For the avoidance of doubt, we would mention that Drosophila and vertebrate names are used interchangeably and that both (all homologues) are included within the scope of the invention.

Key targets for Notch-dependent transcriptional activation are genes of the Enhancer of split complex (E[spl]). Moreover these genes have been shown to be direct targets for binding by the Su(H) protein and to be transcriptionally activated in response to Notch signalling. By analogy with EBNA2, a viral coactivator protein that interacts with a mammalian Su(H) homologue CBF1 to convert it from a transcriptional repressor to a transcriptional activator, the Notch intracellular domain, perhaps in association with other proteins may combine with Su(H) to contribute an activation domain that allows Su(H) to activate the transcription of E(spl) as well as other target genes. It should also be noted that Su(H) is not required for all Notch-dependent decisions, indicating that Notch mediates some cell fate choices by associating with other DNA-binding transcription factors or be employing other mechanisms to transduce extracellular signals.

According to one aspect of the present invention the second sequence is the Notch polypeptide or polynucleotide or a fragment thereof which retains the signalling transduction ability of Notch or an analogue of Notch which has the signalling transduction ability of Notch. By Notch, we mean Notch-1 (as set forth in FIG. 5), Notch-2 (as set forth in FIG. 6), Notch-3, Notch-4 and any other Notch homologues or analogues. In a particularly preferred embodiment the second amino acid sequence is the Notch intracellular domain (Notch IC) or a sub-fragment thereof. For example, the sequence may be a sequence comprising or coding for at least amino acids 1848 to 2202 of human Notch1 , or a sequence having at least 70%, preferably at least 75%, preferably at least 80%, preferably at least 85%, preferably at least 90%, preferably at least 95% sequence homology to such a sequence.

Suitably the sequence comprises an Ankyrin repeat domain and optionally an LNR domain, RAM domain, OPA domain and/or PEST sequence.

As used herein the term “analogue of Notch” includes variants thereof which retain the signalling transduction ability of Notch. By “analogue” we include a protein which has Notch signalling transduction ability, but generally has a different evolutionary origin to Notch. Analogues of Notch include proteins from the Epstein Barr virus (EBV), such as EBNA2, BARF0 or LMP2A.

By a polypeptide or polynucleotide which is for Notch signalling activation we mean a molecule which is capable of activating Notch, the Notch signalling pathway or any one or more of the components of the Notch signalling pathway.

In one embodiment, the molecule for Notch signalling activation will be a dominant negative version of a Notch signalling repressor. In an alternative embodiment, the molecule for Notch signalling activation will be capable of inhibiting a Notch signalling repressor. In a further alternative embodiment, the molecule for Notch signalling activation will be a positive activator of Notch signalling.

Preferably, the molecule will be a polypeptide selected from Noggin, Chordin, Follistatin, Xnr3, fibroblast growth factors and derivatives, fragments, variants and homologues thereof, or a polynucleotide encoding any one or more of the above.

In another embodiment, the molecule may be a Notch ligand, or a polynucleotide encoding a Notch ligand. Notch ligands of use in the present invention include endogenous Notch ligands which are typically capable of binding to a Notch receptor polypeptide present in the membrane of a variety of mammalian cells, for example hematopoietic stem cells.

Particular examples of mammalian Notch ligands identified to date include the Delta family, for example Delta or Delta-like 1 (Genbank Accession No. AF003522—Homo sapiens), Delta-3 (Genbank Accession No. AF084576—Rattus norvegicus) and Delta-like 3 (Mus musculus) (Genbank Accession No. NM_{—}016941—Homo sapiens) and US 6121045 (Millennium), Delta-4 (Genbank Accession Nos. AB043894 and AF 253468—Homo sapiens) and the Serrate family, for example Serrate-1 and Serrate-2 (WO97/01571, WO96/27610 and WO92/19734), Jagged-1 (Genbank Accession No. U73936—Homo sapiens) and Jagged-2 (Genbank Accession No. AF029778—Homo sapiens), and LAG-2. Homology between family members is extensive.

In a preferred embodiment, the activator will be a constitutively active Notch receptor or Notch intracellular domain, or a polynucleotide encoding such a receptor or intracellular domain.

In an alternative embodiment, the activator of Notch signalling will act downstream of the Notch receptor. Thus, for example, the activator of Notch signalling may be a constitutively active Deltex polypeptide or a polynucleotide encoding such a polypeptide. Other downstream components of the Notch signalling pathway of use in the present invention include Deltex-1, Deltex-2, Deltex-3, Suppressor of Deltex (SuDx), Numb and isoforms thereof, Numb associated Kinase (NAK), Notchless, Dishevelled (Dsh), emb5, Fringe genes (such as Radical, Lunatic and Manic), PON, LNX, Disabled, Numblike, Nur77, NFkB2, Mirror, Warthog, Engrailed-1 and Engrailed-2, Lip-1 and homologues thereof, the polypeptides involved in the Ras/MAPK cascade modulated by Deltex, polypeptides involved in the proteolytic cleavage of Notch such as Presenilin and polypeptides involved in the transcriptional regulation of Notch target genes, preferably in a constitutively active form, and analogues, derivatives, variants and fragments thereof.

By polypeptides or polynucleotides for Notch signalling activation is also meant any polypeptides expressed as a result of Notch activation and any polypeptides involved in the expression of such polypeptides, or polynucleotides encoding for such polypeptides.

Activation of Notch signalling may also be achieved by repressing inhibitors of the Notch signalling pathway. As such, polypeptides for Notch signalling activation will include molecules capable of repressing any Notch signalling inhibitors. Preferably the molecule will be a polypeptide, or a polynucleotide encoding such a polypeptide, that decreases or interferes with the production or activity of compounds that are capable of producing a decrease in the expression or activity of Notch, Notch ligands, or any downstream components of the Notch signalling pathway. In a preferred embodiment, the molecules will be capable of repressing polypeptides of the Toll-like receptor protein family and growth factors such as the bone morphogenetic protein (BMP), BMP receptors and activins, derivatives, fragments, variants and homologues thereof.

By a polypeptide or polynucleotide which is for Notch signalling inhibition, we mean a molecule which is capable of inhibiting Notch, the Notch signalling pathway or any one or more of the components of the Notch signalling pathway.

In one embodiment, the molecule for Notch signalling inhibition will be a dominant negative version of a compound capable of activating or transducing Notch signalling. In an alternative. embodiment, the molecule for Notch signalling inhibition will be capable of repressing a compound capable of activating or transducing Notch signalling. In a further alternative embodiment, the molecule for Notch signalling inhibition will be an inhibitor of Notch signalling.

In a particular embodiment, the molecule will be capable of reducing or preventing Notch or Notch ligand expression. Such a molecule may be a nucleic acid sequence capable of reducing or preventing Notch or Notch ligand expression.

Preferably the nucleic acid sequence encodes a polypeptide selected from Toll-like receptor protein family, or a growth factor such as a bone morphogenetic protein (BMP), a BMP receptor and activins. Preferably the agent is a polypeptide, or a polynucleotide encoding such a polypeptide, that decreases or interferes with the production of compounds that are capable of producing an increase in the expression of Notch ligand, such as Noggin, Chordin, Follistatin, Xnr3, fibroblast growth factors and derivatives, fragments, variants and homologues thereof.

Alternatively, the nucleic acid sequence is an antisense construct derived from a sense nucleotide sequence encoding a polypeptide selected from a Notch ligand and a polypeptide capable of upregulating Notch ligand expression, such as Noggin, Chordin, Follistatin, Xnr3, fibroblast growth factors and derivatives, fragments, variants and homologues thereof.

In another preferred embodiment the inhibitor of Notch signalling is a molecule which is capable of modulating Notch-Notch ligand interactions. A molecule may be considered to modulate Notch-Notch ligand interactions if it is capable of inhibiting the interaction of Notch with its ligands, preferably to an extent sufficient to provide therapeutic efficacy.

In this embodiment the molecule may be a polypeptide, or a polynucleotide encoding such a polypeptide, selected from a Toll-like receptor or a growth factor such as a BMP, a BMP receptor and activins. Preferably the polypeptide decreases or interferes with the production of an agent that is capable of producing an increase in the expression of Notch ligand, such as Noggin, Chordin, Follistatin, Xnr3, fibroblast growth factors and derivatives, fragments, variants, homologues and analogs thereof.

Preferably when the inhibitor is a receptor or a nucleic acid sequence encoding a receptor, the receptor is activated. Thus, for example, when the agent is a nucleic acid sequence, the receptor is constitutively active when expressed.

Inhibitors of Notch signalling also include downstream inhibitors of the Notch signalling pathway (such as Dsh and Numb), compounds that prevent expression of Notch target genes or induce expression of genes repressed by the Notch signalling pathway and dominant negative versions Notch signalling transducer molecules (such as of Notch IC and Deltex). Proteins for Notch signalling inhibition will also include variants of the wild-type components of the Notch signalling pathway which have been modified in such a way that their presence blocks rather than transduces the signalling pathway. An example of such a compound would be a Notch receptor which has been modified such that proteolytic cleavage of its intracellular domain is no longer possible.

The modulator may also be an antibody or a nucleotide sequence coding for an antibody. The term “antibody” includes intact molecules as well as fragments thereof, such as Fab, F(ab′)2, Fv and scFv which are capable of binding the epitopic determinant. These antibody fragments retain some ability to selectively bind with its antigen or receptor and include, for example:

(i) Fab, the fragment which contains a monovalent antigen-binding fragment of an antibody molecule can be produced by digestion of whole antibody with the enizyme papain to yield an intact light chain and a portion of one heavy chain;

(ii) Fab′, the fragment of an antibody molecule can be obtained by treating whole antibody with pepsin, followed by reduction, to yield an intact light chain and a portion of the heavy chain; two Fab′ fragments are obtained per antibody molecule;

(iii) F(ab′)₂, the fragment of the antibody that can be obtained by treating whole antibody with the enzyme pepsin without subsequent reduction; F(ab′)₂ is a dimer of two Fab′ fragments held together by two disulfide bonds;

(iv) scFv, including a genetically engineered fragment containing the variable region of a heavy and a light chain as a fused single chain molecule.

General methods of making these fragments are known in the art. (See for example, Harlow and Lane, Antibodies: A, Laboratory Manual, Cold Spring Harbor Laboratory, New York (1988), which is incorporated herein by reference).

The transport proteins used in the present invention are membrane translocation proteins capable of translocating the cell membrane and, in a preferred embodiment, also the nuclear membrane. It is irrelevant whether the protein translocates from the exterior of the cell/nucleus or from the interior, i.e. the proteins may originate within the cytoplasm or nucleus (for example, by virtue of having being synthesised there or inserted into that compartment), and translocate to a location exterior to the cellular compartment (cytoplasm or nucleus) it originates from. In general, the proteins are prepared outside the cell and translocate from an exterior location to the cytoplasm and then optionally, on into the nucleus.

As used herein, the term “cell membrane translocation” refers to the ability of the protein to cross the cell membrane and enter the cytosol/cytoplasm of a cell or to cross from the cytosol/cytoplasm of a cell to the exterior, extra-cellular or interstitial space.

The term “nuclear membrane translocation” refers to the ability of the protein to cross the membrane structure surrounding the cell nucleus, or to cross from the cytosol/cytoplasm of a cell to the nucleus.

In use, many of the products described herein can be expressed as fusion proteins in a first part of the target population of cells, exported therefrom, and taken up by a second part of the target population of cells not directly producing the protein. The first and second part cells may be the same or different cell types. Also within the invention are mammalian and microbial host cells comprising such vectors or other polynucleotides encoding the fusion proteins, and their production and use.

A fusion polypeptide as described herein can be transported to a target population of cells, by introducing a polynucleotide or other vector encoding the fusion polypeptide into a first part of the target population of cells, e.g. by transfection or microinjection; expressing the encoding polynucleotide to produce the fusion polypeptide, thereby to cause it to be exported from said first part of said target population, and to cause it to be taken up by a second part of the target population of cells not directly producing the fusion polypeptide.

Coupled products can also be transported into a target population of cells by directly exposing the cells to a preparation of the coupled products, thereby to cause the target cells to take them up.

Examples of preferred transport proteins include VP22, Antennapedia and HIV tat.

The terms “polypeptide” and “protein” are used interchangeably and refer to endogenous, modified, synthetic and/or natural sequences including variants, derivatives, analogues and fragments thereof. The term “polynucleotide” refers to an endogenous, modified, synthetic and/or natural chain of nucleotides (i.e. DNA or RNA) which may comprise, for example, a protein-encoding domain, an antisense sequence or a functional motif such as a protein-binding domain and includes variants, derivatives, analogues and fragments thereof. The term also refers to polypeptides encoded by the nucleotide sequence.

Within the definitions of “proteins” useful in the present invention, the specific amino acid residues may be modified in such a manner that the protein in question retains at least one of its endogenous functions, such modified proteins are referred to as “variants”. A variant protein can be modified by addition, deletion and/or substitution of at least one amino acid present in the naturally-occurring protein.

Typically, amino acid substitutions may be made, for example from 1, 2 or 3 to 10 or 20 substitutions provided that the modified sequence retains the required transport activity or ability to modulate Notch signalling. Amino acid substitutions may include the use of non-naturally occurring analogues.

The protein used in the present invention may also have deletions, insertions or substitutions of amino acid residues which produce a silent change and result in a functionally equivalent protein. Deliberate amino acid substitutions may be made on the basis of similarity in polarity, charge, solubility, hydrophobicity, hydrophilicity, and/or the amphipathic nature of the residues as long as the transport or modulation function is retained. For example, negatively charged amino acids include aspartic acid and glutamic acid; positively charged. amino acids include lysine and arginine; and amino acids with uncharged polar head groups having similar hydrophilicity values include leucine, isoleucine, valine, glycine, alanine, asparagine, glutamine, serine, threonine, phenylalanine, and tyrosine.

Conservative substitutions may be made, for example according to the Table below. Amino acids in the same block in the second column and preferably in the same line in the third column may be substituted for each other:

| | | |
|---|---|---|
| ALIPHATIC | Non-polar | G A P |
| | | I L V |
| | Polar-uncharged | C S T M |
| | | N Q |
| | Polar-charged | D E |
| | | K R |
| AROMATIC | | H F W Y |

As used herein, the term “protein” includes single-chain polypeptide molecules as well as multiple-polypeptide complexes where individual constituent polypeptides are linked by covalent or non-covalent means. As used herein, the terms “polypeptide” and “peptide” refer to a polymer in which the monomers are amino acids and are joined together through peptide or disulfide bonds. The terms subunit and domain may also refer to polypeptides and peptides having biological function. A peptide usefull in the invention will at least have a transport or signalling modulation capability. “Fragments” are also variants and the term typically refers to a selected region of the protein that is of interest in a binding assay and for which a binding partner is known or determinable. “Fragment” thus refers to an amino acid sequence that is a portion of a full-length polypeptide, between about 8 and about 745 amino acids in length, preferably about 8 to about 300, more preferably about 8 to about 200 amino acids, and even more preferably about 10 to about 50 or 100 amino acids in length. “Peptide” refers to a short amino acid sequence that is 10 to 40 amino acids long, preferably 10 to 35 amino acids.

Such variants may be prepared using standard recombinant DNA techniques such as site-directed mutagenesis. Where insertions are to be made, synthetic DNA encoding the insertion together with 5′ and 3′ flanking regions corresponding to the naturally-occurring sequence either side of the insertion site. The flanking regions will contain convenient restriction sites corresponding to sites in the naturally-occuring sequence so that the sequence may be cut with the appropriate enzyme(s) and the synthetic DNA ligated into the cut. The DNA is then expressed in accordance with the invention to make the encoded protein. These methods are only illustrative of the numerous standard techniques known in the art for manipulation of DNA sequences and other known techniques may also be used.

Variants of the nucleotide sequence may also be made. Such variants will preferably comprise codon optimised sequences. Codon optimisation is known in the art as a method of enhancing RNA stability and therefor gene expression. The redundancy of the genetic code means that several different codons may encode the same amino acid. For example, Leucine, Arginine and Serine are each encoded by six different codons. Different organisms show preferences in their use of the different codons. Viruses such as HIV, for instance, use a large number of rare codons. By changing a nucleotide sequence such that rare codons are replaced by the corresponding commonly used mammalian codons, increased expression of the sequences in mammalian target cells can be achieved. Codon usage tables are known in the art for mammalian cells, as well as for a variety of other organisms.

In one embodiment of the present invention, at least one of the nucleotide sequences encoding either the transport protein or the protein for Notch signalling is codon optimised for expression in mammalian cells.

In a preferred embodiment, the sequences are optimised in their entirety.

The ability of a naturally occurring or synthetic sequence to translocate the membrane or affect Notch signalling may be tested by routine methods known in the art.

Some variants of the known transport proteins which retain the ability to translocate the membrane have been reported in the art and these are included in the scope of the present invention, together with any which become available.

Some variants of the known proteins for Notch signalling modulation which retain this ability have been reported in the art and these are included in the scope of the present invention, together with any which become available.

Preferably, any non-native protein is prepared by use of recombinant techniques.

In a further aspect of the present invention there is provided a polynucleotide sequence encoding the fusion protein of the present invention.

“Polynucleotide” refers to a polymeric form of nucleotides of at least 10 bases in length and up to 1,000 bases or even more, either ribonucleotides or deoxyribonucleotides or a modified form of either type of nucleotide. The term includes single and double stranded forms of DNA.

These may be constructed using standard recombinant DNA methodologies. The nucleic acid may be RNA or DNA and is preferably DNA. Where it is RNA, manipulations may be performed via cDNA intermediates. Generally, a nucleic acid sequence encoding the first region will be prepared and suitable restriction sites provided at the 5′ and/or 3′ ends. Conveniently the sequence is manipulated in a standard laboratory vector, such as a plasmid vector based on pBR322 or pUC19 (see below). Reference may be made to Molecular Cloning by Sambrook et al. (Cold Spring Harbor, 1989) or similar standard reference books for exact details of the appropriate techniques.

Nucleic acid encoding the second region may likewise be provided in a similar vector system.

Sources of nucleic acid may be ascertained by reference to published literature or databanks such as GenBank. Nucleic acid encoding the desired first or second sequences may be obtained from academic or commercial sources where such sources are willing to provide the material or by synthesising or cloning the appropriate sequence where only the sequence data are available. Generally this may be done by reference to literature sources which describe the cloning of the gene in question.

Alternatively, where limited sequence data is available or where it is desired to express a nucleic acid homologous or otherwise related to a known nucleic acid, exemplary nucleic acids can be characterised as those nucleotide sequences which hybridise to the nucleic acid sequences known in the art. Genes will be referred to interchangeably under their human or Drosophila nomenclature. Reference to a gene is meant to include the gene itself and any, homologues thereof.

It will be understood by a skilled person that numerous different nucleotide sequences can encode the same transport protein or protein for Notch signalling modulation used in the present invention as a result of the degeneracy of the genetic code. In addition, it is to be understood that skilled persons may, using routine techniques, make nucleotide substitutions that do not affect the transport protein or protein for Notch signalling modulation encoded by the nucleotide sequence of the present invention to reflect the codon usage of any particular host organism in which the transport protein or protein for Notch signalling, modulation of the present invention is to be expressed.

In general, the terms “variant”, “homologue” or “derivative” in relation to the nucleotide sequence used in the present invention includes any substitution of, variation of, modification of, replacement of, deletion of or addition of one (or more) nucleic acid from or to the sequence providing the resultant nucleotide sequence codes for a transport protein or protein for Notch signalling modulation.

As indicated above, with respect to sequence homology, preferably there is at least 75%, more preferably at least 85%, more preferably at least 90% homology to the reference sequences. More preferably there is at least 95%, more preferably at least 98%, homology. Nucleotide homology comparisons may be conducted as described above. A preferred sequence comparison program is the GCG Wisconsin Bestfit program described above. The default scoring matrix has a match value of 10 for each identical nucleotide and −9 for each mismatch. The default gap creation penalty is −50 and the default gap extension penalty is −3 for each nucleotide.

The present invention also encompasses nucleotide sequences that are capable of hybridising selectively to the reference sequences, or any variant, fragment or derivative thereof, or to the complement of any of the above. Nucleotide sequences are preferably at least 15 nucleotides in length, more preferably at least 20, 30, 40 or 50 nucleotides in length.

The term “hybridization” as used herein shall include “the process by which a strand of nucleic acid joins with a complementary strand through base pairing” as well as the process of amplification as carried out in polymerase chain reaction (PCR) technologies.

Nucleotide sequences useful in the invention capable of selectively hybridising to the nucleotide sequences presented herein, or to their complement, will be generally at least 75%, preferably at. least 85 or 90% and more preferably at least 95% or 98% homologous to the corresponding nucleotide sequences presented herein over a region of at least 20, preferably at least 25 or 30, for instance at least 40, 60 or 100 or more contiguous nucleotides.

The term “selectively hybridizable” means that the nucleotide se quence used as a probe is used under conditions where a target nucleotide sequence of the invention is found to hybridize to the probe at a level significantly above background. The background hybridization may occur because of other nucleofide sequences present, for example, in the cDNA or genomic DNA library being screened. In this event, background implies a level of signal generated by interaction between the probe and a non-specific DNA member of the library which is less than 10 fold, preferably less than 100 fold as intense as the specific interaction observed with the target DNA. The intensity of interaction may be measured, for example, by radiolabelling the probe, e.g. with ³²P.

Hybridization conditions are based on the melting temperature (Tm) of the nucleic acid binding complex, as taught in Berger and Kimmel (1987, Guide to Molecular Cloning Techniques, Methods in Enzymology, Vol 152, Academic Press, San Diego Calif.), and confer a defined “stringency” as explained below.

Maximum stringency typically occurs at about Tm−5° C. (5° C. below the Tm of the probe); high stringency at about 5° C. to 10° C. below Tm; intermediate stringency at about 10° C. to 20° C. below Tm; and low stringency at about 20° C. to 25° C. below Tm. As will be understood by those of skill in the art, a maximum stringency hybridization can be used to identify or detect identical nucleotide sequences while an intermediate (or low) stringency hybridization can be used to identify or detect similar or related polynucleotide sequences.

In a preferred aspect, the present invention covers nucleotide sequences that can hybridise to the nucleotide sequence of the present invention under stringent conditions (e.g. 65° C. and 0.1×SSC {1×SSC=0.15 M NaCl, 0.015 M Na₃ Citrate pH 7.0). Where the nucleotide sequence of the invention is double-stranded, both strands of the duplex, either individually or in combination, are encompassed by the present invention. Where the nucleotide sequence is single-stranded, it is to be understood that the complementary sequence of that nucleotide sequence is also included within the scope of the present invention.

Nucleotide sequences which are not 100% homologous to the sequences of the present invention but fall within the scope of the invention can be obtained in a number of ways. Other variants of the sequences described herein may be obtained for example by probing DNA libraries made from a range of sources. In addition, other viral/bacterial, or cellular homologues particularly cellular homologues found in mammalian cells (e.g. rat, mouse, bovine and primate cells), may be obtained and such homologues and fragments thereof in general will be capable of selectively hybridising to the sequences shown in the sequence listing herein. Such sequences may be obtained by probing cDNA libraries made from or genomic DNA libraries from other animal species, and probing such libraries with probes comprising all or part of the reference nucleotide sequence under conditions of medium to high stringency. Similar considerations apply to obtaining species homologues and allelic variants of the amino acid and/or nucleotide sequences useful in the present invention.

Variants and strain/species homologues may also be obtained using degenerate PCR which will use primers designed to target sequences within the variants and homologues encoding conserved amino acid sequences within the sequences of the present invention. Conserved sequences can be predicted, for example, by aligning the amino acid sequences from several variants/homologues. Sequence alignments can be performed using computer software known in the art. For example the GCG Wisconsin PileUp program is widely used. The primers used in degenerate PCR will contain one or more degenerate positions and will be used at stringency conditions lower than those used for cloning sequences with single sequence primers against known sequences.

Alternatively, such nucleotide sequences may be obtained by site directed mutagenesis of characterised sequences. This may be useful where for example silent codon changes are required to sequences to optimise codon preferences for a particular host cell in which the nucleotide sequences are being expressed. Other sequence changes may be desired in order to introduce restriction enzyme recognition sites, or to alter the activity of the transport protein or protein for Notch signalling modulation encoded by the nucleotide sequences.

The nucleotide sequences such as a DNA polynucleotides useful in the invention may be produced recombinantly, synthetically, or by any means available to those of skill in the art. They may also be cloned by standard techniques.

In general, primers will be produced by synthetic means, involving a step wise manufacture of the desired nucleic acid sequence one nucleotide at a time. Techniques for accomplishing this using automated techniques are readily available in the art.

Longer nucleotide sequences will generally be produced using recombinant means, for example using a PCR (polymerase chain reaction) cloning techniques. This will involve making a pair of primers (e.g. of about 15 to 30 nucleotides) flanking a region of the targeting sequence which it is desired to clone, bringing the primers into contact with MRNA or cDNA obtained from an animal or human cell, performing a polymerase chain reaction (PCR) under conditions which bring about amplification of the desired region, isolating the amplified fragment (e.g. by purifying the reaction mixture on an agarose gel) and recovering the amplified DNA. The priners may be designed to contain suitable restriction enzyme recognition sites so that the amplified DNA can be cloned into a suitable cloning vector

According to further aspects of the present invention there is provided an expression vector comprising the polynucleotide sequence of the present invention; a host cell transformed with the expression vector of the present invention; a method for preparing a fusion protein of the present invention comprising culturing the host cell of the present invention under conditions which provide for the expression of the fusion protein; a method of transporting a protein for Notch signalling modulation into a cell comprising exposing a cell to a conjugate according to the present invention; a conjugate prepared by the method of the present invention; a pharmaceutical composition comprising the conjugate of the present invention, particularly for use in the treatment of T-cell mediated disease; and use of the conjugate of the present invention in the preparation of a medicament for the prevention and/or treatment of disease or infection, particularly a T-cell mediated disease.

### BRIEF DESCRIPTION OF THE DRAWINGS

The following Detailed Description, given by way of example, but not intended to limit the invention to specific embodiments described, may be understood in conjunction with the accompanying drawings, incorporated herein by reference. Various preferred features and embodiments of the present invention will now be described by way of non-limiting example and with reference to the accompanying drawings in which:

FIG. 1, comprising FIGS. 1A-1D, shows a schematic representation of Notch;

FIG. 2 shows a schematic representation of NotchIC;

FIGS. 3 and 4 show schematic representations of the Notch signalling pathway;

FIG. 5 shows the amino acid sequence of human Notch 1 (GenBank AF308602) (SEQ ID NO:11);

FIG. 6 shows the amino acid sequence of human Notch 2 (GenBank AAA36377) (SEQ ID NO:12);

FIG. 7, comprising FIGS. 7A and 7B, shows the results of Example 4(iii), and is a comparison of transactivation of reporter vectors with NIC2202;

FIG. 8 shows the results of Example 5(ii) and relates to co-tansfection of C2C12 cells with mHes1-Luc and Notch IC expression vectors;

FIG. 9 shows the results of Example 7 and relates to transfection of luceriferase reporter vectors into stable CHO cell clones;

FIG. 10 shows results from Example 8 and relates to the co-culture of stable CHO cell clones with C2C12 cells transfected with mHes1-Luc for 48 hours; and

FIG. 11 shows results from Example 8 and relates to the co-culture of stable CHO cell clones with C2C12 cells transfected with mHes1-Luc : time course experiment.

### DETAILED DESCRIPTION

The practice of the present invention will employ, unless otherwise indicated, conventional techniques of chemistry, molecular biology, microbiology, recombinant DNA and immunology, which are within the capabilities of a person of ordinary skill in the art. Such techniques are explained in the literature. See, for example, J. Sambrook, E. F. Fritsch, and T. Maniatis, 1989, Molecular Cloning: A Laboratory Manual, Second Edition, Books 1-3, Cold Spring Harbor Laboratory Press; Ausubel, F. M. et al. (1995 and periodic supplements; Current Protocols in Molecular Biology, ch. 9, 13, and 16, John Wiley & Sons, New York, N.Y.); B. Roe, J. Crabtree, and A. Kahn, 1996, DNA Isolation and Sequencing: Essential Techniques, John Wiley & Sons; J. M. Polak and James O'D. McGee, 1990, In Situ Hybridization: Principles and Practice; Oxford University Press; M. J. Gait (Editor), 1984, Oligonucleotide Synthesis: A Practical Approach, Irl Press; and, D. M. J. Lilley and J. E. Dahlberg, 1992, Methods of Enzymology: DNA Structure Part A: Synthesis and Physical Analysis of DNA Methods in Enzymology, Academic Press. Each of these general texts is herein incorporated by reference.

### Transport Protein

Herpesvirus VP22 Protein (VP22)

The herpesvirus (HSV) VP22 protein (VP22) is a 38 kDa protein which as described in WO97/05625 and Elliott & O'Hare shows a transport function. VP22 is the product of the HSV1 gene, UL49 (GenBank X14112). VP22 is transported from the cytoplasm of an expressing cell where it accumulates in the nucleus of neighbouring cells. In addition, VP22 is taken up be unexpressing cells where it accumulates in the nucleus. Thus fusion of the VP22 sequence to an amino acid sequence or polynucleotide sequence can provide a useful cell delivery vector.

In this'specification, ‘VP22’ denotes: protein VP22 of HSV, e.g. of HSV1, and transport-active fragments and homologues thereof, including transport active homologues from other herpesviruses including varicella zoster virus VZV, equine herpesvirus EHV and bovine herpesvirus BHV; and variant proteins having a transport function corresponding to a transport function of VP22 of HSV1.

Among fragments of herpesvirus VP22 protein with transport activity WO97/05265 reports that transport activity is present in polypeptides corresponding to amino acids (aa) 60-301 and 159-301 of the full HSV1 VP22 sequence (aa 1-301).

Accordingly, the present invention relates in one aspect to coupled and fusion proteins comprising a sub-sequence of VP22 containing a sequence starting preferable from about aa 159 (or earlier, towards the N-terminal, in the native VP22 sequence), to about aa 301, and having (relative to the full VP22 sequence) at least one deletion of at least part of the VP22 sequence which can extend for example from the N-terminal to the cited starting point, e.g. a deletion of all or part of the sequence of about aa 1-158.

VP22 sequences as contemplated herein extend to homologous proteins and fragments based on sequences of VP22 protein homologues from other herpesviruses, e.g. the invention provides corresponding derivatives and uses of the known VP22-homologue sequences from VZV (e.g. all or homologous parts of the sequence from aa 1-302), from MDV (e.g. all or homologous parts of the sequence from aa 1-249) and from BHV (e.g. all or homologous parts of the sequence from aa 1-258). The sequences of the corresponding proteins from HS V2, VZV, SHV and MDV are available in public protein/nucleic acid sequence databases. Thus, for example, within the EMBL/Genbank database, a VP22 sequence from HSV2 is available as gene item UL29 under accession no. Z86099 containing the complete genome of HSV2 strain HG52; the complete genome of VZV including the homologous gene/protein is available under accession numbers X04370, M14891. M16612; the corresponding protein sequence from BHV is available as ‘bovine herpesvirus 1 virion tegument protein’ under accession number U21137; and the corresponding sequence from MDV is available as gene item UL49 under accession number L10283 for ‘gallid herpesvirus type 1 homologous sequence genes’. In these proteins, especially those from HSV2 and VZV, corresponding deletions can be made, e.g. of sequences homologous to aa 1-159 of VP22 from HSV1.

Furthermore, chimeric VP22 proteins and protein sequences are also useful within the context of the present invention, e.g. a protein sequence from VP22 of HSV1 for part of which a homologous sequence from the corresponding VP22 homologue of another herpesvirus has been substituted. For example, into the sequence of polypeptide 159-301 from VP22 of HSV1, C-terminal sequences can be substituted from VP22 of HSV2 or from the VP22 homologue of BHV.

Homeodomains

It has been proposed that homeodomains in general may be used as efficient transduction vectors (Jeon) and thus may be used in the present invention.

Homeoproteins are trans-activating factors involved in multiple morphological processes. They bind to DNA through a sequence of 60 amino acid residues, the so-called homeodomain. The structure of this domain consists of three α-helices, interrupted by a β-turn between helices 2 and 3 (Gehring et al.). The phylogenetic relationship between numerous homeoproteins is striking at the level of the homeodomain and particularly within the third α-helix. This helix is responsible for both the interaction with DNA, as well as the capacity of homeoproteins to translocate across cell membranes to cell nuclei in a non-specific manner. Examples of homoedomains which may be used in preferred embodiments of the present invention are the hoinoeodomains of Antennapedia, Fushi-tatzu and Engrailed.

In a particularly preferred embodiment the homoedomain is that of Antennapedia. In more detail, the gene antennapedia (Antp) encodes a transcriptional factor that has been shown to control antero-posterior morphogenesis in Drosophila embryo. The protein sequence of antennapedia is characterised by the presence of a 60 amino acids motif (homeodomain) that binds to specific DNA target elements. Antennapedia homologues have been found in nearly all multicellular organisms and show a very high degree of amino acid sequence identity. The human and Drosophila antennapedia proteins differ in the sequence of the homeodomain only for one conservative amino acid substitution.

It has been observed that antennapedia and its homeodomain are able to translocate across the cytoplasmic membrane of mammalian cells. The translocation does not depend on cell endocytosis and it has been reported that translocation occurs at both 4° C. and 37° C. Homeodomain synthetic peptides made of D amino acids are also able to cross the cytoplasmic membrane. This finding would rule out the possibility that Antp is translocated through a receptor mediated mechanism. This property has been exploited to vehiculate small viral sequences into the cytoplasm of cultured cells as well as to elicit an MHC class I restricted cytotoxic immune response against the nucleoprotein of the influenza virus.

The homeodomain of the Antp gene preferably is obtainable from Drosophila. Sequences homologous to this homeodomain have been isolated from other organisms, including vertebrates, mammals and humans, and these are included in the present invention. The homeodomain may be prepared using standard techniques such as cloning. As previously indicated differences in the sequences of such multicellular organisms are generally conservative in nature. However, this may not necessarily be the case and other such sequences are included in the present invention, and for example where the sequence identity is about 50% or more, e.g. 60%, 70%, 80% or 90%, with the sequence obtainable from Drosophila.

European Patent 485578 discloses that the homeodomain and specifically, helix 3 of a homeobox peptide, particularly that derived from the Drosophila Antennapedia, is of use as an intracellular transport vector. The patent disclosed that a specific 57 amino acid sequence of a Drosophila Antennapedia homeopeptide (referred to as the pAntp peptide) was capable of penetrating fibroblasts and embryo cells (in vivo). Emphasis was placed upon the last 27 amino acids of the sequence that correspond with the helix 3 and 4.

Subsequent disclosures (Derossi D et al. (1994), Derossi D et al. (1996), Joliot et al., Perez F et al.) have focused on a 16 amino acid synthetic peptide derived from the third helix of the Antennapedia homeodomain that may be used for the intracellular delivery of bioactive products and antisense oligonucle6tides. The amino acid sequence of this peptide is RQIKIWFQNRRMKWKK (SEQ ID NO:13), also known as penetratin. In the course of their investigations the above authors synthesized several variants on this sequence, these corresponding to residues 41-60, 41-55 and 46-60 of the pAntp peptide and showed that in all cases, the only peptides to intemalise into the cells were those that included the residues 43-58 (Derossi D et al., supra.).

In an effort to prevent the enzymatic cleavage of this peptide Brigidou. J et al., prepared a retro-inverso form (D amino acids in reverse order) substituting the two isoleucine resides at, positions 3 and 5 of penetratin with valine and adding a glycine residue at the C-terminus to facilitate binding to a resin. A further retro-inverso form was prepared replacing the extra glycine with a cholesterol moiety attached via a sulfhydryl linker group. The addition of the cholesterol moiety improved penetration due to the increased hydrophobicity of the molecule.

This development of the retro-inverso form of penetratin has given rise to WO97/12912 that. discloses peptides of 16 amino acids comprising between 6 and 10 hydrophobic amino acids wherein the sixth amino acid from either end must be tryptophan. This disclosure attempts to define the minimal characteristics of sequences capable of acting as intemalisation vectors as being the retention of a tryptophan residue at the sixth position from the amino terminus and, that the peptide contains from 6 to 10 hydrophobic amino acid residues (the classification of hydrophobic amino residues in WO97/12912 is not believed to be in agreement with the generally accepted classification).

From the disclosures discussed above, as summarised in WO97/12912, it has been concluded that essential to the membrane translocating properties of the homeodomain peptides, is the presence of a tryptophan residue as the sixth residue from the amino terminus. Conforming to these requirements has been a penetratin variant of the formula (KWKK)₄ which has been described as having translocating ability that discloses a number of branched membrane translocating peptides such as (KWKK)₂KGGC, wherein each KWKK is joined to the following lysine residue.

Further variants are disclosed in WO00/29427, having a formula:

| RQIKIWFQNRRMKWKK | (SEQ ID NO:13) |
|---|---|
| 1 16 | |

wherein at least one amino acid residue is deleted from the amino terminus, or variants thereof. Thus, it has been observed that the ability to translocate a cell membrane is retained with sequences not containing the whole of residues 43-58 of the pAntp peptide.

HIV tat Protein

Purified human immunodeficiency virus type1 (“HIV”) tat protein is taken up from the surrounding medium by human cells growing in culture (A. D. Frankel and C. O. Pabo). Tat protein trans-activates certain HIV genes and is essential for viral replication. The full-length HIV-1 tat protein has 86 amino acid residues. The HIV tat gene has two exons. Tat amino acids 1-72 are encoded by exon 1, and amino acids 73-86 are encoded by exon 2. The. full-length tat protein is characterised by a basic region which contains two lysines and six arginines (amino acids 49-57) and a cystein-rich region which contains seven cysteine residues (amino acids 22-237).

The basic region (i.e. amino acids 49-57) is thought to be important for nuclear localization. (Ruben, S. et al.; Hauber, J. et al.). The cysteine-rich region mediates the formation of metal-linked dimers in vitro (Frankel, A. D. et .al., and Frankel, A. D. et al.) and is essential for its activity as a transactivator (Garcia, J. A. et al. And Sadaie, M. R. et al.). As in other regulatory proteins, the N-terminal region may be involved in protection against intracellular proteases (Bachmair, A. et al).

The preferred transport polypeptides of this invention are characterized by the presence of the tat basic region amino acid sequence (amino acids 49-57 of naturally-occurring tat protein); the absence of the tat cysteine-rich region amino acid sequence (amino acids 22-36 of naturally-occurring tat protein) and the absence of the tat exon 2-encoded carboxy-terminal domain (amino acids 73-86 of naturally-occurring tat protein). Preferred embodiments of such transport polypeptides are: tat37-72, tat37-58, tat38-58GGC, tatCGG47-58, tat47-58GGC, and tatAcys. It will be recognised by those of ordinary skill in the art that when the transport polypeptide is genetically fused to the cargo moiety, an amino-terminal methionine must be added, but the spacer amino acids (e.g., CysGlyGly or GlyGlyCys) need not be added.

### Protein for Notch Signalling Modulation

a. Polypeptides and Polynucleotides for Notch Signalling Transduction:

The Notch signalling pathway directs binary cell fate decisions in the embryo. Notch was first described in Drosophila as a transmembrane protein that functions as a receptor for two different ligands, Delta and Serrate. Vertebrates express multiple Notch receptors and ligands (discussed below). At least four Notch receptors (Notch-1, Notch-2, Notch-3 and Notch-4) have been identified to date in human cells.

Notch proteins are synthesized as single polypeptide precursors that undergo cleavage via a Furin-like convertase that yields two polypeptide chains that are further processed to form the mature receptor. The Notch receptor present in the plasma membrane comprises a heterodimer of two Notch proteolytic cleavage products, one comprising an N-terminal fragment consisting of a portion of the extracellular domain, the transmembrane domain and the intracellular domain, and the other comprising the majority of the extracellular domain. The proteolytic cleavage step of Notch to activate the receptor occurs and is mediated by a furin-like convertase.

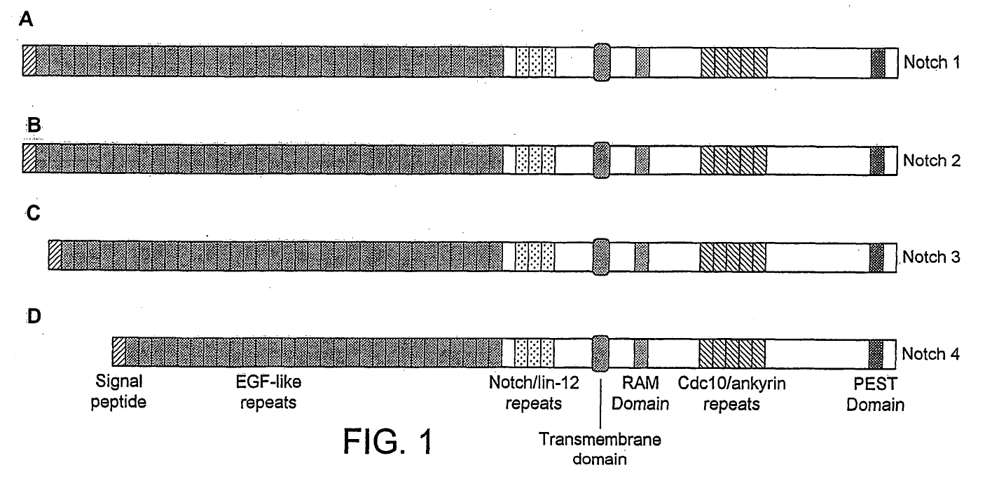

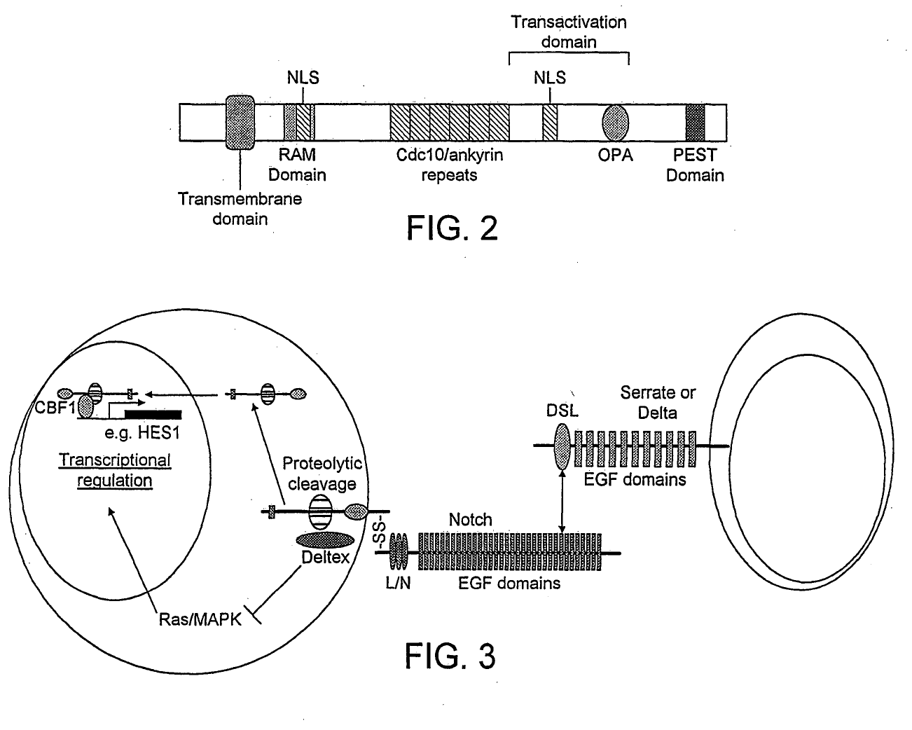

Notch receptors are inserted into the membrane as disulphide-linked heterodimeric molecules consisting of an extracellular domain containing 36 epidermal growth factor (EGF)-like repeats and a transmembrane subunit that contains the cytoplasmic domain. The cytoplasmic domain of Notch contains six ankyrin-like repeats, a polyglutamine stretch (OPA) and a PEST sequence. A further domain termed RAM23 lies proximal to the ankyrin repeats and is involved in binding to a transcription factor, known as Suppressor of Hairless [Su(H)] in Drosophila and CBF1 in vertebrates (Tamura). The Notch ligands also display multiple EGF-like repeats in their extracellular domains together with a cysteine-rich DSL (Delta-Serrate Lag2) domain that is characteristic of all Notch ligands (Artavanis-Tsakonas). Schematic representations of Notch and the Notch intracellular domain are shown in FIGS. 1 and 2.

The Notch receptor is activated by binding of extracellular ligands, such as Delta, Serrate and Scabrous, to the EGF-like repeats of Notch's extracellular domain. Delta requires cleavage for activation. It is cleaved by the ADAM disintegrin metalloprotease Kuzbanian at the cell surface, the cleavage event releasing a soluble and active form of Delta. An oncogenic variant of the human Notch-1 protein, also known as TAN-1, which has a truncated extracellular domain, is constitutively active and has been found to be involved in T-cell. lymphoblastic leukemias.

The cdc10/ankyrin intracellular-domain repeats mediate physical interaction with intracellular signal transduction proteins. Most notably, the cdc10/ankyrin repeats interact with Suppressor of Hairless [Su(H)]. Su(H) is the Drosophila homologue of C-promoter binding factor-1 [CBF-1], a mammalian DNA binding protein involved in the Epstein-Barr virus-induced immortalization of B-cells. It has been demonstrated that, at least in cultured cells, Su(H) associates with the cdc10/ankyrin repeats in the cytoplasm and translocates into the nucleus upon the interaction of the Notch receptor with its ligand Delta on adjacent cells. Su(H) includes responsive elements found in the promoters of several genes and has been found to be a critical downstream protein in the Notch signalling pathway. The involvement of Su(H) in transcription is thought to be modulated by Hairless.

The intracellular domain of Notch (NotchIC) also has a direct nuclear function (Lieber). Recent studies have indeed shown that Notch activation requires that the six cdc10/ankyrin repeats of the Notch intracellular domain reach the nucleus and participate in transcriptional activation. The site of proteolytic cleavage on the intracellular tail of Notch has been identified between gly1743 and val1744 (termed site 3, or S3) (Schroeter). It is thought that the proteolytic cleavage step that releases the cdc10/ankyrin repeats for nuclear entry is dependent on Presenilin activity.

The intracellular domain has been shown to accumulate in the nucleus where it forms a transcriptional activator complex with the CSL family protein CBF1 (suppressor of hairless, Su(H) in Drosophila, Lag-2 in C. elegans) (Schroeter; Struhl). The NotchIC-CBF1 complexes then activate target genes, such as the bHLH proteins HES (hairy-enhancer of split like) 1 and 5 (Weinmaster). This nuclear function of Notch has also been shown for the mammalian Notch homologue (Lu).

Processing of the Notch intracellular domain occurs only in response to binding of Notch ligands Delta or Serrate/Jagged. The post-translational modification of the nascent Notch receptor in the Golgi (Munro; Ju) appears, at least in part, to control which of the two types of ligand is expressed on a cell surface. The Notch receptor is modified on its extracellular domain by Fringe, a glycosyl transferase enzyme that binds to the Notch/Lin motif. Fringe modifies Notch by adding O-linked fucose groups to the EGF-like repeats (Moloney; Bruckner). This modification by Fringe does not prevent ligand binding, but may influence ligand induced conformational changes in Notch. Furthermore, recent studies suggest that the action of Fringe modifies Notch to prevent it from interacting functionally with Serrate/Jagged ligands but allow it to preferentially bind Delta (Panin; Hicks). Although Drosophila has a single Fringe gene, vertebrates are known to express multiple genes (Radical, Manic and Lunatic Fringes), (Irvine).

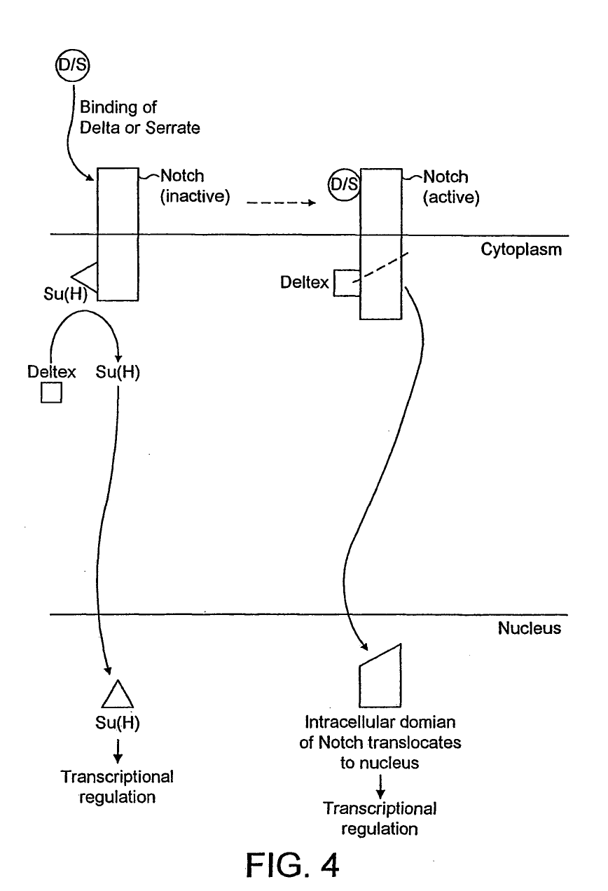

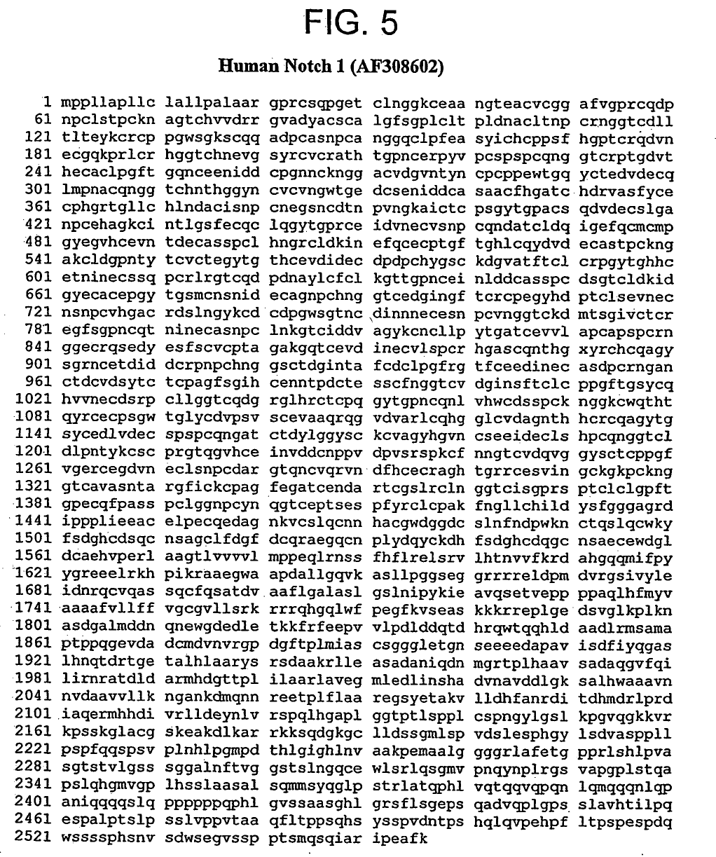

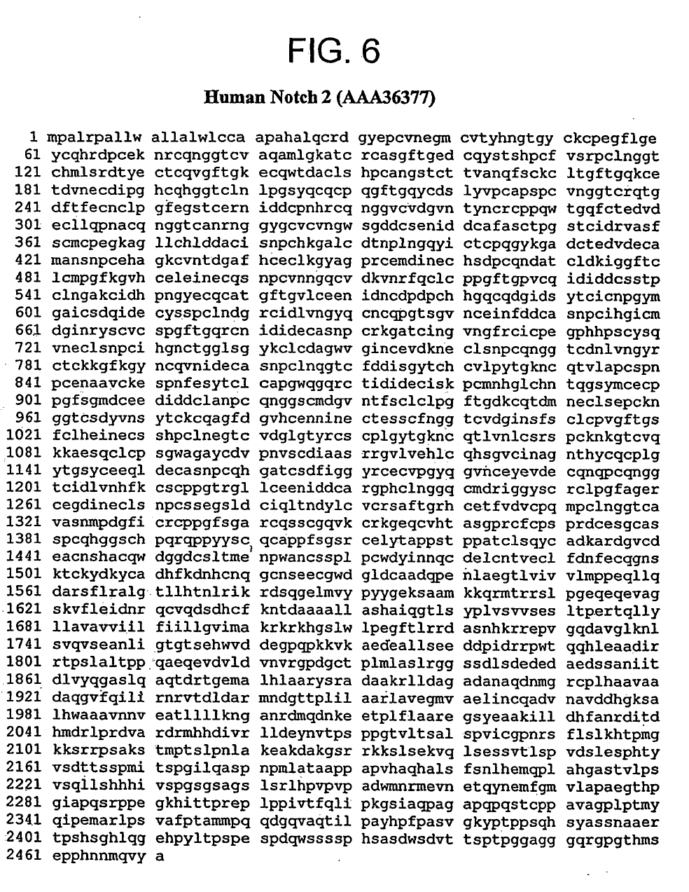

Thus, signal transduction from the Notch receptor can occur via two different pathways both of which are illustrated in FIG. 3 and 4. Target genes of the Notch signalling pathway include Deltex, genes of the Hes family (Hes-1 in particular), Enhancer of Split [E(spl)] complex genes, IL-10, CD-23, CD-4 and Dll-1.

Deltex, an intracellular docking protein, replaces Su(H) as it leaves its site of interaction with the intracellular tail of Notch, as shown in FIG. 3. Deltex is a cytoplasmic protein containing a zinc-finger (Artavanis-Tsakonas; Osborne). It interacts with the ankyrin repeats of the Notch intracellular domain. Studies indicate that Deltex promotes Notch pathway activation by interacting with Grb2 and modulating the Ras-JNK signalling pathway (Matsuno). Deltex also acts as a docking protein which prevents Su(H) from binding to the intracellular tail of Notch (Matsuno). Thus, Su(H) is released into the nucleus where it acts as a transcriptional modulator. Recent evidence also suggests that, in a vertebrate B-cell system, Deltex, rather than the Su(H) homologue CBF1, is responsible, for inhibiting E47 function (Ordentlich). Expression of Deltex is upregulated as a result of Notch activation in a positive feedback loop. The sequence of Homo sapiens Deltex (DTX1) mRNA may be found in GenBank Accession No. AF053700.

Hes-1 (Hairy/Enhancer of Split-1) (Takebayashi) is a transcriptional factor with a basic helix-loop-helix structure. It binds to an important functional site in the CD4 silencer leading to repression of CD4 gene expression. Thus, Hes-1 is strongly involved in the determination of T-cell fate. Other genes from the Hes family include Hes-5 (mammalian Enhancer of Split homologue), the expression of which is also upregulated by Notch activation, and Hes-3. Expression of Hes-1 is upregulated as a result of Notch activation. The sequence of Hes-1 can be found in GenBank Accession Nos. AK000415 and AV264785.

The E(spl) gene complex [E(spl)-C] (Leimeister) comprises seven genes of which only E(spl) and Groucho show visible phenotypes when mutant. E(spl) was named after its ability to enhance Split mutations, Split being another name for Notch. Indeed, E(spl)-C genes repress Delta through regulation of achaete-scute complex gene expression. Expression of E(spl) is upregulated as a result of Notch activation.

IL-10 (interleukin-10) is a factor produced by Th2 helper T-cells and regulatory T-cells. It is a co-regulator of mast cell growth and shows extensive homology with the Epstein-Barr bcrfi gene. Although it is not known to be a direct downstream target of the Notch signalling pathway, its expression has been found to be strongly upregulated coincident with Notch activation and its promoter has several CBF1 response elements. The MRNA sequence of IL-10 may be found in GenBank ref. No. GI1041812.

CD-23 is the human leukocyte differentiation antigen CD23 (FCE2) which is a key molecule for B-cell activation and growth. It is the low-affinity receptor for IgE. Furthermore, the truncated molecule can be secreted, then functioning as a potent mitogenic growth factor. Although it is not thought to be a direct downstream target of the Notch signalling pathway, its expression has been found to be strongly upregulated by Notch activation. The sequence for CD-23 may be found in GenBank ref. No. GI1783344.

CTLA4 (cytotoxic T-lymphocyte activated protein 4) is an accessory molecule found on the surface of T-cells which is thought to play a role in the regulation of airway inflammatory cell recruitment and T-helper cell differentiation after allergen inhalation. The promoter region of the gene encoding CTLA4 has CBF1 response elements and its expression is upregulated as a result of Notch activation. The sequence of CTLA4 can be found in GenBank Accession No. L15006.

Dlx-1 (distalless-1) (McGuiness) expression is downregulated as a result of Notch activation. Sequences for Dlx genes may be found in GenBank Accession Nos. U51000-3.

CD-4 expression is downregulated as a result of Notch activation. A sequence for the CD-4 antigen may be found in GenBank Accession No. XM006966.

Other genes involved in the Notch signaling pathway, such as Numb, Mastermind and Dsh, and all genes the expression of which is modulated by Notch activation, are included in the scope of this invention.

Analogues of NotchIC

As described above the Notch receptor family participates in cell-cell signalling events that influence T cell fate decisions. In this signalling NotchIC localises to the nucleus and functions as an activated receptor. Mammalian NotchIC interacts with the transcriptional repressor CBF1. It has been proposed that the NotchIC cdc10/ankyrin repeats are essential for this interaction. Hsieh et al suggests rather that the N-terminal 114 amino acid region of mouse NotchIC contains the CBF1 interactive domain. It is also proposed that NotchIC acts by targeting DNA-bound CBF1 within the nucleus and abolishing CBF1-mediated repression through masking of the repression domain. It is known that Epstein Barr virus (EBV) immortalizing protein EBNA” also utilises CBF1 tethering and masking of repression to upregulate expression of CBF1-repressed B-cell genes. Thus, mimicry of Notch signal transduction is involved in EBV-driven immortalization. Strobl et al similarly reports that “EBNA2 may hence be regarded as a functional equivalent of an activated Notch receptor”. Other EBV proteins which fall in this category include BARF0 (Kusano and Raab-Traub) and LMP2A.

b. Polypeptides and Polynucleotides for Notch Signalling Activation:

Examples of mammalian Notch ligands identified to date include the Delta family, for example Delta-1 (Genbank Accession No. AF003522—Homo sapiens), Delta-3 (Genbank Accession No. AF084576—Rattus norvegicus) Delta-like 3 (Mus musculus) and Delta-4 (Genbank Accession No. AB043894), the Serrate family, for example Serrate-1 and Serrate-2 (WO97/01571, WO96/27610 and WO92/19734), Jagged-1 and Jagged-2 (Genbank Accession No. AF029778—Homo sapiens), and LAG-2. Homology between family members is extensive. For example, human Jagged-2 has 40.6% identity and 58.7% similarity to Serrate.

Further homologues of known mammalian Notch ligands may be identified using standard techniques. By a “homologue” it is meant a gene product that exhibits sequence homology, either amino acid or nucleic acid sequence homology, to any one of the known Notch ligands, for example as mentioned above. Typically, a homologue of a known Notch ligand will be at least 20%, preferably at least 30%, identical at the arnino acid level to the corresponding known Notch ligand. Techniques and software for calculating sequence homology between two or more amino acid or nucleic acid sequences are well known in the art (see for example http://www.ncbi.nlm.nih.gov and Ausubel et al., Current Protocols in Molecular Biology (1995), John Wiley & Sons, Inc.)

Notch ligands identified to date have a diagnostic DSL domain (D. Delta, S. Serrate, L. Lag2) comprising 20 to 22 amino acids at the amino terminus of the protein and between 3 to 8 EGF-like repeats on the extracellular surface. It is therefor preferred that homologues of Notch ligands also comprise a DSL domain at the N-terminus and between 3 to 8 EGF-like repeats on the extracellular surface.

In addition, suitable homologues will be capable of binding to a Notch receptor. Binding may be assessed by a variety of techniques known in the art including in vitro binding assays.

Homologues of Notch ligands can be identified in a number of ways, for example by probing genomic or cDNA libraries with probes comprising all or part of a nucleic acid encoding a Notch ligand under conditions of medium to high stringency (for example 0.03M sodium chloride and 0.03M sodium citrate at from about 50° C. to about 60° C.). Alternatively, homologues may also be obtained using degenerate PCR which will generally use primers designed to target sequences within the variants and homologues encoding conserved amino acid sequences. The primers will contain one or more degenerate positions and will be used at stringency conditions lower than those used for cloning sequences with single sequence primers against known sequences.

Other substances capable of activating the Notch signalling pathway include compounds capable of upregulating Notch ligand expression including polypeptides that bind to and reduce or neutralise the activity of bone morphogenetic proteins (BMPs). Binding of extracellular BMPs (Wilson and Hemmati-Brivanlou, Hemmati-Brivanlou and Melton) to their receptors leads to down-regulated Delta transcription due to the inhibition of the expression of transcription factors of the achaete/scute complex. This complex is believed to be directly involved in the regulation of Delta expression. Thus, any substance that inhibits BMP expression and/or inhibits the binding of BMPs to their receptors may be capable of producing an increase in the expression of Notch ligands such as Delta and/or Serrate. Particular examples of such inhibitors include Noggin (Valenzuela), Chordin (Sasai), Follistatin (Iemura), Xnr3, and derivatives and variants thereof. Noggin and Chordin bind to BMPs thereby preventing activation of their signalling cascade which leads to decreased Delta transcription. Consequently, increasing Noggin and Chordin levels may lead to increase Notch ligand, in particular Delta, expression.

Furthermore, any substance that upregulates expression of transcription factors of the achaete/scute complex may also upregulate Notch ligand expression.

Other suitable substances that may be used to upregulate Notch ligand expression include transforming growth factors such as members of the fibroblast growth factor (FGF) family. The FGF may be a mammalian basic FGF, acidic° FGF or another member of the FGF family such as an FGF-1, FGF-2, FGF-3, FGF-4, FGF-5, FGF-6, FGF-7. Preferably the FGF is not acidic FGF (FGF-1; Zhao). Most preferably, the FGF is a member of the FGF family which acts by stimulating the upregulation of expression of a Serrate polypeptide on APCs. It has been shown that members of the FGF family can upregulate Serrate-1 gene expression in APCs.

The substance capable of upregulating expression of Notch or a Notch ligand may be selected from polypeptides and fragments thereof, linear peptides, cyclic peptides, including synthetic and natural compounds. The substances capable of upregulating expression of a Notch ligand may be derived from a biological material such as a component of extracellular matrix. Suitable extracellular matrix components are derived from immunologically privileged sites such as the eye. For example aqueous humour or components thereof may be used.

Polypeptide substances such as Noggin, FGFs and TGF-β may be purified from mammalian cells, obtained by recombinant expression in suitable host cells or obtained commercially. Alternatively, nucleic acid constructs encoding the polypeptides may be used. As a further example, overexpression of Notch or Notch ligand, such as Delta or Serrate, may be brought about by introduction of a nucleic acid construct capable of activating the endogenous gene, such as the Serrate or Delta gene. In particular, gene activation can be achieved by the use of homologous recombination to insert a heterologous promoter in place of the natural promoter, such as the Serrate or Delta promoter, in the genome of the target cell.

The activating molecule of the present invention will, in an alternative embodiment, be capable of modifying Notch-protein expression or presentation on the cell membrane or signalling pathways. Agents that enhance the presentation of a fully functional Notch-protein on the target cell surface include matrix metalloproteinases such as the product of the Kuzbanian gene of Drosophila (Dkuz) and other ADAMALYSIN gene family members.

C. Polypeptides and Polynucleotides for Notch Signalling Inhibition

Substances that may be used to inhibit Notch ligand expression include nucleic acid sequences encoding polypeptides that affect the expression of genes encoding Notch ligands. For instance, for Delta expression, binding of extracellular BMPs (bone morphogenetic proteins, Wilson and Hemmati-Brivanlou; Hemmati-Brivanlou and Melton) to their receptors leads to down-regulated Delta transcription due to the inhibition of the expression of transcription factors of the achaete/scute complex. This complex is believed to be directly involved in the regulation of Delta expression. Thus, any polypeptide that upregulates BMP expression and/or stimulates the binding of BMPs to their receptors may be capable of producing a decrease in the expression of Notch ligands such as Delta and/or Serrate. Examples may include nucleic acids encoding BMPs themselves. Furthermore, any substance that inhibits expression of transcription factors of the achaete/scute complex may also downregulate Notch ligand expression.

Members of the BMP family include BMP1 to BMP6, BMP7 also called OP1, OP2 (BMP8) and others. BMPs belong to the transforming growth factor beta (TGF-beta) superfamily, which includes, in addition to the TGF-betas, activins/inhibins (e.g., alpha-inhibin), mullerian inhibiting substance, and glial cell line-derived neurotrophic factor.

Other examples of polypeptides that inhibit the expression of Delta and/or Serrate include the Toll-like receptor (Medzhitov) or any other receptors linked to the innate immune system (for example CD14, complement receptors, scavenger receptors or defensin proteins), and other polypeptides that decrease or interfere with the production of Noggin (Valenzuela), Chordin (Sasai), Follistatin (lemura), Xnr3, and derivatives and variants thereof (Hoyne). Noggin and Chordin bind to BMPs thereby preventing activation of their signalling cascade which leads to decreased Delta transcription. Consequently, reducing Noggin and Chordin levels may lead to decrease Notch ligand, in particular Delta, expression (Hoyne et al, 2000).

In more detail, in Drosophila, the Toll transmembrane receptor plays a central role in the signalling pathways that control amongst other things the innate nonspecific immune response. This Toll-mediated immune response reflects an ancestral conserved signalling system that has homologous components in a wide range of organisms. Human Toll homologues have been identified amongst the Toll-like receptor (TLR) genes and Toll/interleukin-1 receptor-like (TIL) genes and contain the characteristic Toll motifs: an. extracellular leucine-rich repeat domain and a cytoplasmic interleukin-1 receptor-like region. The Toll-like receptor genes (including TIL genes) now include TLR4, TIL3, TIL4, and 4 other identified TLR genes.

Other suitable sequences that may be used to downregulate Notch ligand expression include those encoding immune costimrulatory molecules (for example CD80, CD86, ICOS, SLAM) and other accessory molecules that are associated with immune potentiation (for example CD2, LFA-1).

Other suitable substances that may be used to downregulate Notch ligand expression include nucleic acids that inhibit the effect of transforming growth factors such as members of the fibroblast growth factor (FGF) family. The FGF may be a mammalian basic FGF, acidic FGF or another member of the FGF family such as an FGF-1, FGF-2, FGF-3, FGF-4, FGF-5, FGF-6, FGF-7. Preferably the FGF is not acidic FGF (FGF-1; Zhao et al., 1995). Most preferably, the FGF is a member of the FGF family which acts by stimulating the upregulation of expression of a Serrate polypeptide on APCs. The inventors have shown that members of the FGF family can upregulate Serrate-1 gene expression in APCs.

Suitable nucleic acid sequences may include anti-sense constructs, for example nucleic acid sequences encoding antisense Notch ligand constructs as well as antisense constructs designed to reduce or inhibit the expression of upregulators of Notch ligand expression (see above). The antisense nucleic acid may be an oligonucleotide such as a synthetic single-stranded DNA. However, more preferably, the antisense is an antisense RNA produced in the patient's own cells as a result of introduction of a genetic vector. The vector is responsible for production of antisense RNA of the desired specificity on introduction of the vector into a host cell.

Preferably, the nucleic acid sequence for use in the present invention is capable of inhibiting Serrate and Delta, preferably Serrate 1 and Serrate 2 as well as Delta 1, Delta 3 and Delta 4 expression in APCs such as dendritic cells. In particular, the nucleic acid sequence may be capable of inhibiting Serrate expression but not Delta expression in APCs. Alternatively, the nucleic acid sequence for use in the present invention is capable of inhibiting Delta expression in T cells such as CD4⁺ helper T cells or other cells of the immune system that express Delta (for example in response to stimulation of cell surface receptors). In particular, the nucleic acid sequence may be capable of inhibiting Delta expression but not Serrate expression in T cells. In a particularly preferred embodiment, the nucleic acid sequence is capable of inhibiting Notch ligand expression in both T cells and APC, for example Serrate: expression in APCs and Delta expression in T cells.

Preferred suitable substances that may be used to downregulate Notch ligand expression include growth factors and cytokines. More preferably soluble protein growth factors may be used to inhibit Notch or Notch ligand expression. For instance, Notch ligand expression may be reduced or inhibited by the addition of BMPs or activins (a member of the TGF-β superfarnily). In addition, T cells, APCs or tumour cells could be cultured in the presence of inflammatory type cytokines including IL-12, IFN-γ, IL-18, TNF-α, either alone or in combination with BMPs.

Molecules for inhibition of Notch signalling will also include polypeptides, or polynucleotides which encode therefor, capable of modifying Notch-protein expression or presentation on the cell membrane or signalling pathways. Molecules that reduce or interfere with its presentation as a fully functional cell membrane protein may include MMP inhibitors such as hydroxymate-based inhibitors.

Other substances which may be used to reduce interaction between Notch and Notch ligands are exogenous Notch or Notch ligands or functional derivatives thereof. Such Notch ligand derivatives would preferably have the DSL domain at the N-terminus and between 3 to 8 EGF-like repeats on the extracellular surface. A peptide corresponding to the Delta/Serrate/LAG-2 domain of hJagged1 and supernatants from COS cells expressing a soluble form of the extracellular portion of hJagged1 was found to mimic the effect of Jagged1 in inhibiting Notch1 (Li).

Whether a substance can be used for modulating Notch-Notch ligand expression may be determined using suitable screening assays.

Notch signalling can be monitored either through protein assays or through nucleic acid assays. Activation of the Notch receptor leads to the proteolytic cleavage of its cytoplasmic domain and the translocation thereof into the cell nucleus. The “detectable signal” referred to herein may be any detectable manifestation attributable to the presence of the cleaved intracellular domain of Notch. Thus, increased Notch signalling can be assessed at the protein level by measuring intracellular concentrations of the cleaved Notch domain. Activation of the Notch receptor also catalyses a series of downstream reactions leading to changes in the levels of expression of certain well defined genes. Thus, increased Notch signalling can be assessed at the nucleic acid level by say measuring intracellular concentrations of specific mRNAs. In one preferred embodiment of the present invention, the assay is a protein assay. In another preferred embodiment of the present invention, the assay is a nucleic acid assay.

The advantage of using a nucleic acid assay is that they are sensitive and that small samples can be analysed.

The intracellular concentration of a particular mRNA, measured at any given time, reflects the level of expression of the corresponding gene at that time. Thus, levels of mRNA of downstream target genes of the Notch signalling pathway can be measured in an indirect assay of the T-cells of the immune system. In particular, an increase in levels of Deltex, Hes-1 and/or ILL-10 mRNA may, for instance, indicate induced anergy while an increase in levels of Dll-1 or IFN-(mRNA, or in the levels of mRNA encoding cytokines such as IL-2, IL-5 and IL-13, may indicate improved responsiveness.

Various nucleic acid assays are known. Any convention technique which is known or which is subsequently disclosed may be employed. Examples of suitable nucleic acid assay are mentioned below and include amplification, PCR, RT-PCR, RNase protection, blotting, spectrometry, reporter gene assays, gene chip arrays and other hybridization methods.

In particular, gene presence, amplification and/or expression may be measured in a sample directly, for example, by conventional Southern blotting, Northern blotting to quantitate the transcription of mRNA, dot blotting (DNA or RNA analysis), or in situ hybridisation, using an appropriately labelled probe. Those skilled in the art will readily envisage how these methods may be modified, if desired.

PCR was originally developed as a means of amplifying DNA from an impure sample. The technique is based on a temperature cycle which repeatedly heats and cools the reaction solution allowing primers to anneal to target sequences and extension of those primers for the formation of duplicate daughter strands. RT-PCR uses an RNA template for generation of a first strand cDNA with a reverse transcriptase. The cDNA is then amplified according to standard PCR protocol. Repeated cycles of synthesis and denaturation result in an exponential increase in the number of copies of the target DNA produced. However, as reaction components become limiting, the rate of amplification decreases until a plateau is reached and there is little or no net increase in PCR product. The higher the starting copy number of the nucleic acid target, the sooner this “end-point” is reached.

Real-time PCR uses probes labeled with a fluorescent tag or fluorescent dyes and differs from end-point PCR for quantitative assays in that it is used to detect PCR products as they accumulate rather than for the measurement of product accumulation after a fixed number of cycles. The reactions are characterized by the point in time during cycling when amplification of a target sequence is first detected through a significant increase in fluorescence.

The ribonuclease protection (RNase protection) assay is an extremely sensitive technique for the quantitation of specific RNAs in solution. The ribonuclease protection assay can be performed on total cellular RNA or poly(A)-selected mRNA as a target. The sensitivity of the ribonuclease protection assay derives from the use of a complementary in vitro transcript probe which is radiolabeled to high specific activity. The probe and target RNA are hybridized in solution, after which the mixture is diluted and treated with ribonuclease (RNase) to degrade all remaining single-stranded RNA. The hybndized portion of the probe will be protected from digestion and can be visualized via electrophoresis of the mixture on a denaturing polyacrylamide gel followed by autoradiography. Since the protected fragments are analyzed by high resolution polyacrylamide gel electrophoresis, the ribonuclease protection assay can be employed to accurately map mRNA features. If the probe is hybridized at a molar excess with respect to the target RNA, then the resulting signal will be directly proportional to the amount of complementary RNA in the sample.

Gene expression may also be detected using a reporter system. Such a reporter system may comprise a readily identifiable marker under the control of an expression system, e.g. of the gene being monitored. Fluorescent markers, which can be detected and sorted by FACS, are preferred. Especially preferred are GFP and luciferase. Another type of preferred reporter is cell surface markers, ie. proteins expressed on the cell surface and therefor easily identifiable.

In general, reporter constructs useful for detecting Notch signalling by expression of a reporter gene may be constructed according to the general teaching of Sambrook et al (1989). Typically, constructs according to the invention comprise a promoter by the gene of interest, and a coding sequence encoding the desired reporter constructs, for example of GFP or luciferase. Vectors encoding GFP and luciferase are known in the art and available commercially.

Sorting of cells, based upon detection of expression of genes, may be performed by any technique known in the art, as exemplified above. For example, cells may be sorted by flow cytometry or FACS. For a general reference, see Flow Cytometry and Cell Sorting: A Laboratory Manual (1992) A. Radbruch (Ed.), Springer Laboratory, New York.

Flow cytometry is a powerful method for studying and purifying cells. It has found wide application, particularly in immunology and cell biology: however, the capabilities of the FACS can be applied in many other fields of biology. The acronym F.A.C.S. stands for Fluorescence Activated Cell Sorting, and is used interchangeably with “flow cytometry”. The principle of FACS is that individual cells, held in a thin stream of fluid, are passed through one or more laser beams, causing light to be scattered and fluorescent dyes to emit light at various frequencies. Photomultiplier tubes (PMT) convert light to electrical signals, which are interpreted by software to generate data about the cells. Sub-populations of cells with defined characteristics can be identified and automatically sorted from the suspension at very high purity (˜100%).

FACS can be used to measure gene expression in cells transfected with recombinant DNA encoding polypeptides. This can be achieved directly, by labelling of the protein product, or indirectly by using a reporter gene in the construct. Examples of reporter genes are β-galactosidase and Green Fluorescent Protein (GFP). β-galactosidase activity can be detected by FACS using fluorogenic substrates such as fluorescein digalactoside (FDG). FDG is introduced into cells by hypotonic shock, and is cleaved by the enzyme to generate a fluorescent product, which is trapped within the cell. One enzyme can therefor generate a large amount of fluorescent product. Cells expressing GFP constructs will fluoresce without the addition of a substrate. Mutants of GFP are available which have different excitation frequencies, but which emit fluorescence in the same channel. In a two-laser FACS machine, it is possible to distinguish cells which are excited by the different lasers and therefor assay two transfections at the same time.

Alternative means of cell sorting may also be employed. For example, the invention comprises the use of nucleic acid probes complementary to mRNA. Such probes can be used to identify cells expressing polypeptides individually, such that they may subsequently be sorted either manually, or using FACS sorting. Nucleic acid probes complementary to mRNA may be prepared according to the teaching set forth above, using the general procedures as described by Sambrook et al (1989).

In a preferred embodiment, the invention comprises the use of an antisense nucleic acid molecule, complementary to a mRNA, conjugated to a fluorophore which may be used in FACS cell sorting.

Methods have also been described for obtaining information about gene expression and identity using so-called gene chip arrays or high density DNA arrays (Chee). These high density arrays are particularly useful for diagnostic and prognostic purposes. Use may also be made of In Vivo Expression Technology (IVET) (Camilli). IVET identifies genes up-regulated during say treatment or disease when compared to laboratory culture.

The advantage of using a protein assay is that Notch activation can be directly measured. Assay techniques that can be used to determine levels of a polypeptide are well known to those skilled in the art. Such assay methods include radioimmunoassays, competitive-binding assays, Western Blot analysis, antibody sandwich assays, antibody detection, FACS and ELISA assays.

### Preperation Expression Vectors and Host Cells

The conjugates of the present invention may be prepared by any methods known in the art.

The present invention also relates to vectors which comprise a polynucleotide useful in the present invention, host cells which are genetically engineered with vectors of the invention and the production of polypeptides useful in the present invention by such techniques.

For recombinant production, host cells can be genetically engineered to incorporate expression systems or polynucleotides of the invention. Introduction of a polynucleotide into the host cell can be effected by methods described in many standard laboratory manuals, such as Davis et al and Sambrook et al, such as calcium phosphate transfection, DEAE-dextran mediated transfection, transvection, microinjection, cationic lipid-mediated transfection, electroporation, transduction, scrape loading, ballistic introduction and infection.

Representative examples of appropriate hosts include bacterial cells, such as streptococci, staphylococci, E. coli, streptomyces and Bacillus subtilis cells; fungal cells, such as yeast cells and Aspergillus cells; insect cells such as Drosophila S2 and Spodoptera Sf9 cells; animal cells such as CHO, COS, NSO, HeLa, C127, 3T3, BHK, 293 and Bowes melanoma cells; and plant cells.

A great variety of expression systems can be used to produce a polypeptide useful in the present invention. Such vectors include, among others, chromosomal, episomal and virus-derived vectors, e.g., vectors derived from bacterial plasmids, from bacteriophage, from transposons, from yeast episomes, from insertion elements, from yeast chromosomal elements, from viruses such as baculoviruses, papova viruses, such as SV40, vaccinia viruses, adenoviruses, fowl pox viruses, pseudorabies viruses and retroviruses, and vectors derived from combinations thereof, such as those derived from plasmid and bactenrophage genetic elements, such as cosmids and phagemids. The expression system constructs may contain control regions that regulate as well as engender expression. Generally, any system or vector suitable to maintain, propagate or express polynucleotides and/or to express a polypeptide in a host may be used for expression in this regard. The appropriate DNA sequence may be inserted into the expression system by any of a variety of well-known and routine techniques, such as, for example, those set forth in Sambrook et al.

For secretion of the translated protein into the lumen of the endoplasmic reticulum, into the periplasmic space or into the extracellular environment, appropriate secretion signals may be incorporated into the expressed polypeptide. These signals may be endogenous to the polypeptide or they may be heterologous signals.

Conjugates of the invention can be recovered and purified from recombinant cell cultures by well-known methods including ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. Most preferably, high performance liquid chromatography is employed for purification. Well known techniques for refolding protein may be employed to regenerate active conformation when the polypeptide is denatured during isolation and/or purification.

Chemically coupled sequences can be prepared from individual proteins sequences and coupled using known chemically coupling techniques. The conjugate can be assembled using conventional solution- or solid-phase peptide synthesis methods, affording a fully protected precursor with only the terminal amino group in deprotected reactive form. This function can then be reacted directly with a protein for Notch signalling modulation or a suitable reactive derivative thereof. Alternatively, this amino group may be converted into a different functional group suitable for reaction with a cargo moiety or a linker. Thus, e.g. reaction of the amino group with succinic anhydride will provide a selectively addressable carboxyl group, while further peptide chain extension with a cysteine derivative will result in a selectively addressable thiol group. Once a suitable selectively addressable functional group has been obtained in the delivery vector precursor, a protein for Notch signalling modulation or a derivative thereof may be attached through e.g. amide, ester, or disulphide bond formation. Cross-linking reagents which can be utilized are discussed, for example, in Neans, G. E. and Feeney, R. E., Chemical Modification of Proteins, Holden-Day, 1974, pp. 39-43.

As discussed above the transport protein and protein for Notch signalling modulation may be linked directly or indirectly via a cleavable linker moiety. Direct linkage may occur through any convenient functional group on the protein for Notch signalling modulation such as a hydroxy, carboxy or amino group. Indirect linkage which is preferable, will occur through a linking moiety. Suitable linking moieties include bi- and multi-functional alkyl, aryl, aralkyl or peptidic moieties, alkyl, aryl or aralkyl aldehydes acids esters and anyhdrides, sulphydryl or carboxyl groups, such as maleimido benzoic acid derivatives, maleimido proprionic acid derivatives and succinimido derivatives or may be derived from cyanuric bromide or chloride, carbonyldiimidazole, succinimidyl esters or sulphonic halides and the like. The functional groups on the linker moiety used to form covalent bonds between linker and protein for Notch signalling modulation on the one hand, as well as linker and transport protein on the other hand, may be two or more of, e.g., amino, hydrazino, hydroxyl, thiol, maleimido, carbonyl, and carboxyl groups, etc. The linker moiety may include a short sequence of from 1 to 4 amino acid residues that optionally includes a cysteine residue through which the linker moiety bonds to the transport protein.

In accordance with the present invention each transport protein may be linked to at least one protein for Notch signalling modulation. In a further embodiment, the transport protein is prepared such as to facilitate linkage to more than one protein for Notch signalling modulation, each protein for Notch signalling modulation being the same or different. For example, the transport protein may comprise components that themselves facilitate the: attachment of more than one protein for Notch signalling modulation such as derivatives of naturally occurring amino acids or insertion of a multi-valent synthetic amino acid, or it may be specfically adapted to do so for example by a network of branched lysine residues that may be attached to the transport protein as a linking group and each lysine residue may then be attached to a protein for Notch signalling modulation. In this manner a, single transport protein may carry up to 32 proteins for Notch signalling modulation, preferably from 2 to 10 or more preferably from 4 to 5 proteins for Notch signalling modulation. In this further embodiment each protein for Notch signalling modulation may be directly or indirectly linked to the carrier moiety. When more than one different type of protein for Notch signalling modulaiton is attached, it is possible to co-ordinate the ratios and dosages of the individual drugs to facilitate the administration of specific protein combinations.

Stable aggregates, having particle sizes for example in the range of from 0.1 to 5 microns, may be formed by mixing the protein for Notch signalling modulation with the transport protein. Ratios of from 2:1 to 1:1 of transport protein to protein for Notch signalling modulation are preferred.

In a further embodiment, the conjugate may further comprise a targeting moiety. The targeting moiety is capable of directing the transport protein to the specific cell type to which it is preferable for the protein for Notch signalling modulation to function. Thus, the targeting moiety acts as an address system biasing the body's natural distribution of drugs or the conjugate to a particular cell type. The targeting moiety may be attached to the protein for Notch signalling modulation or more preferably to the transport protein and will direct the conjugate to a desired site, upon arrival at which the transport protein will facilitate the cellular intemalisatidn of the protein for Notch signalling modualtion. Suitable targeting moieties include, for example, cell specific antibodies or antibody fragments such as phage-displayed ScFv and other peptide sequences identified by E Ruoslahti et al. in U.S. Pat. No. 5,622,699; Pasqualini, R, Ruoslahti E; Ruoslahti E; and Arap, W, Pasqualini, R. Ruoslahti, E.

A stabilizing agent, which serves to increase conjugate stability and uptake, can optionally be brought into contact with cells, in conjunction with the conjugate. For example, metal ions which bind to tat protein and increase its stability and uptake, can be used for this purpose.

In a further embodiment of this invention, a lysosomotrophic agent is provided extracellularly in conjunction with the conjugate, in order to enhance uptake by cells. The lysosomotrophic agent can be used alone or in conjunction with a stabilizer. For example lysosomotrophic agents such as chloroquine, monensin, amnantadine and methylamine, which have been shown to increase uptake of tat in some cells by a few hundred fold, can be used for this purpose.

In another embodiment, a basic peptide, such as tat 38-58 or protamine, is provided extracellularly with the conjugate to enhance its uptake. Such basic peptides can also be used alone, in combination or with stabilizing agents or, lysosomotrophic agents.

### Production of Antibodies

The conjugates of the present invention can also be used to raise antibodies which can be used in diagnostic and monitoring specific binding assays using conventional techniques, for example, monitoring the localisation of the conjugates themselves or their components.

In accordance with yet another embodiment of the present invention, there are provided antibodies specifically recognising and binding the conjugates according to the invention. More preferably, however, the antibodies are specific for the second sequence of the. conjugates. Advantageously, the second sequence of the conjugate is recognised by the antibodies when in its natural context. Thus, where the second sequence is an isolated fragment or domain from a protein for Notch signalling modulations that fragment or domain is recognised by the antibodies of the invention in the context of the whole of the larger protein.

The invention moreover provides a method for preparing an immunoglobulin, comprising the steps of:

a) immunsing an animal with a conjugate according to the present invention: and

b) recovering immunoglobulin specific for a region of the conjugate from the serum of the animal.

The antibodies (or immunoglobulins) may be isolated in the form of a crude preparation, i.e. an antiserum, by affinity chromatography against the conjugate. Alternatively, monoclonal antibodies may be prepared according to standard techniques in the art and purified.

### Therapeutic Uses

The therapeutic effect resulting from the administration of the conjugate may arise from the intact conjugate or any of the dissociated proteins for Notch signalling modulation alone or bound to the linker, part of the linker or the linker and part of the transport protein.

A detailed description of the Notch signalling pathway and conditions affected by it may be found in our WO98/20142, WO00/36089 and PCT/GB00/04391.

Diseased or infectious states that may be described as being mediated by T cells include, but are not limited to, any one or more of asthma, allergy, graft rejection, autoimmunity, tumour induced aberrations to the T cell system and infectious diseases such as those caused by Plasmodium species, Microfilariae, Helminths, Mycobacteria, HIV, Cytomegalovirus, Pseudomonas, Toxoplasma, Echinococcus, Haemophilus influenza type B, measles, Hepatitis C or Toxicara. Thus particular conditions that may be treated or prevented which are mediated by T cells include multiple sclerosis, rheumatoid arthritis and diabetes. The present invention may also be used in organ transplantation or bone marrow transplantation.

The present invention is also useful in methods for altering the fate of a cell, tissue or organ type by altering Notch pathway function in the cell. Thus, the present application has application in the treatment of malignant and pre-neoplastic disorders. The present invention is especially useful in relation to adenocarcinomas such as: small cell lung cancer, and cancer of the kidney, uterus, prostrate, bladder, ovary, colon and breast. For example, malignancies which may be treatable according to the present invention include acute and chronic leukemias, lymphomas, myelomas, sarcomas such as Fibrosarcoma, myxosarcoma, liposarcoma, lymphangioendotheliosarcoma, angiosarcoma, endotheliosarcoma, chondrosarcoma, osteogenic sarcoma, chordoma, lymphangiosarcoma, synovioma, mesothelioma, leimybsarcoma, rhabdomyosarcoma, colon carcinoma, ovarian cancer, prostate cancer, pancreatic cancer, breasy cancer, squamous cell carcinoma, basal cell carcinoma, adenocarcinoma, sewat gland carcinoma, sebaceous gland carcinoma, papillary carcinoma, papillary adenocarcinomas, cystadenocarcinoma, medullary carcinoma, bronchogenic carcinoma, choriocarcinoma, renal cell carcinoma, hepatoma, bile duct. carcinoma seminoma, embryonal carcinoma, cervical cancer, testicular tumour, lung carcinoma, small cell lung carcinoma, bladder carcinoma, epithelial carcinoma, glioma, astrocytoma, ependymoma, pinealoma, hemangioblastoma, acoustic neuoma, medulloblastoma, craniopharyngioma, oligodendroglioma, menangioma, melanoma, neutroblastoma and retinoblastoma.

The present invention may also have application in the treatment of nervous system disorders. Nervous system disorders which may be treated according to the present invention include neurological lesions including traumatic lesions resulting from physical injuries; ischaemic lesions; malignant lesions; infectious lesions such as those caused by HIV, herpes zoster or herpes simplex virus, Lyme disease, tuberculosis or syphilis; degenerative lesions and diseases and demyelinated lesions.

The present invention may be used to treat, for example, diabetes (including diabetic neuropathy, Bell's palsy), systemic lupus erythematosus, sarcoidosis, multiple sclerosis, human immunodeficiency virus-associated myelopathy, transverse myelopathy or various etiologies, progressive multifocal leukoencephalopathy, central pontine myelinolysis, Parkinson's disease, Alzheimer's disease, Huntington's chorea, amyotrophic lateral sclerosis, cerebral infarction or ischemia, spinal cord infarction or ischemia, progressive spinal muscular atrophy, progressive bulbar palsy, primary lateral sclerosis, infantile and juvenile muscular atrophy, progressive bulbar paralysis of childhood (Fazio-Londe syndrome), poliomyelitis and the post polio syndrome, and Hereditary Motorsensory Neuropathy (Charcot-Marie-Tooth Disease).

The present invention may further be useful in the promotion of tissue regeneration and repair. The present invention, therefore, may also be used to treat diseases associated with defective tissue repair and regeneration such as, for example, cirrhosis of the liver, hypertrophic scar formation and psoriasis. The invention may also be useful in the treatment of neutropenia or anemia and in techniques of organ regeneration and tissue engineering.

### Methods of Delivery

The present invention can be used to deliver a protein for Notch signal modulation into cells, particularly the cell nucleus, in vitro or in vivo. In in vitro applications, the conjugate may be added to a culture medium of the target cells. The conjugate can also be combined with a cell sample obtained from an individual in order to introduce the protein for Notch signalling modulation into cells present in the sample. After treatment in this manner the sample can be returned to the individual. The conjugate can also be administered in vivo. For example cells that synthesise the conjugate can be produced and implanted into an individual. In a further embodiment, the conjugate can be used much like a conventional therapeutic agent and can be a component of a pharmaceutical composition.

For example, delivery can be carried out in vitro by adding a conjugate to cultured cells, by producing cells that synthesize conjugate or by combining a sample (e.g., blood, bone marrow) obtained from an individual with the conjugate, under appropriate conditions. Thus, the target cells may be in vitro cells, i.e., cultured animals cells, human cells or micro-organisms. Delivery can be carried out in vivo by administering the conjugate to an individual in whom it is to be used for diagnostic, preventative or therapeutic purposes. The target cells may be in vivo cells, i.e., cells composing the organs or tissues of living animals or humans, or microorganisms found in living animals or humans.

### Administration

The conjugates of the present invention are typically formulated for administration to patients with a pharmaceutically acceptable carrier or diluent to produce a pharmaceutical composition. The formulation will depend upon the nature of the compound identified and the route of administration but typically they can be formulated for topical, parenteral, intramuscular, intravenous, intra-peritoneal, intranasal inhalation, lung inhalation, intradermal or intra-articular administration. The conjugate may be used in an injectable form. It may therefor be mixed with any vehicle which is pharmaceutically acceptable for an injectable formulation, preferably for a direct injection at the site to be treated, although it may be administered systemically.

The pharmaceutically acceptable carrier or diluent may be, for example, sterile isotonic saline solutions, or other isotonic solutions such as phosphate-buffered saline. The conjugates of the present invention may be admixed with any suitable binder(s), lubricant(s), suspending agent(s), coating agent(s), solubilising agent(s). It is also preferred to formulate the compound in an orally active form.

In general, a therapeutically effective daily oral or intravenous dose of the conjugate of the invention is likely to range from 0.01 to 50 mg/kg body weight of the subject to be treated, preferably 0.1 to 20 mg/kg. The conjugate may also be administered by intravenous infusion; at a dose which is likely to range from 0.001-10 mg/kg/hr.

Tablets or capsules of the conjugates may be administered singly or two or more at a time, as appropriate. It is also possible to administer the conjugates in sustained release formulations.

Typically, the physician will determine the actual dosage which will be most suitable for an individual patient and it will vary with the age, weight and response of the particular patient. The above dosages are exemplary of the average case. There can, of course, be individual instances where higher or lower dosage ranges are merited, and such are within the scope of this invention.

Alternatively, the conjugates of the invention can be administered by inhalation, intranasally or in the form of aerosol, or in the form of a suppository or pessary, or they may be applied topically in the form of a lotion, solution, cream, ointment or dusting powder. An alternative means of transdermal administration is by use of a skin patch. For example, they can be incorporated into a cream consisting of an aqueous emulsion of polyethylene glycols or liquid paraffin. They can also be incorporated, at a concentration of between 1 and 10% by weight, into an ointment consisting of a white wax or white soft paraffin base together with such stabilisers and preservatives as may be required.

For some applications, preferably the conjugates are administered orally in the form of tablets containing excipients such as starch or lactose, or in capsules or ovules either alone or in admixture with excipients, or in the form of elixirs, solutions or suspensions containing flavouring or colouring agents.

The conjugates can also be injected parenterally, for example intracavemosally, intravenously, intramuscularly or subcutaneously. In this case, the conjugates will comprise a suitable carrier or diluent.

For parenteral administration, the conjugates are best used in the form of a sterile aqueous solution which may contain other substances, for example enough salts or monosaccharides to make the solution isotonic with blood.

For buccal or sublingual administration the conjugates may be administered in the form of tablets or lozenges which can be formulated in a conventional manner.

For oral, parenteral, buccal and sublingual administration to subjects (such as patients), the daily dosage level of the conjugates of the present invention and their pharmaceutically acceptable salts and solvates may typically be from 10 to 500 mg (in single or divided doses). Thus, and by way of example, tablets or capsules may contain from 5 to 100 mg of active compound for administration singly, or two or more at a time; as appropriate. As indicated above, the physician will determine the actual dosage which will be most suitable for an individual patient and it will vary with the age, weight and response of the particular patient. It is to be noted that whilst the above-mentioned dosages are exemplary of the average case there can, of course, be individual instances where higher or lower dosage ranges are merited and such dose ranges are within the scope of this invention.

The routes of administration and dosages described are intended only as a guide since a skilled practitioner will be able to determine readily the optimum route of administration and dosage for any particular patient depending on, for example, the age, weight and condition of the patient.

The term treatment or therapy as used herein should be taken to encompass diagnostic and prophylatic applications.

The treatment of the present invention includes both human and veterinary applications.

The conjugates of the present invention provide several advantages over known delivery systems. These advantages include improved efficacy compared to conventional treatments, improved cellular uptake of the therapeutic agent, improved water solubility, reduction of side effects and cellular bioavailability and decreased occurrence of drug resistance.

The present invention is additionally described by way of the following illustrative, non-limiting Examples, which provide a better understanding of the present invention and of its many advantages.

### EXAMPLES

### Example 1

### Generation of mHES1-Luc Reporter Vector (pLOM3)

A fragment of mouse HES1 promoter fragment (approximately 350 bp) was, generated by PCR from mouse genomic DNA using the following two amplimers:

| | |
|---|---|
| 5′ GGGGTACCCTCAGGCGCGCGCCATTGGCC 3′ | (SEQ ID NO:1) |
| and | |
| 5′ GAAGATCTGCTTACGTCCTTTTACTTGAC 3′ | (SEQ ID NO:2) |

The PCR product was digested with KpnI and BgII, cloned into pGL3-Basic (Promega) and cut with KpnI and BgIII to generate mHES1-Luc-(pLOR3).

### Example 2

### Generation of 4×CBF-1 Reporter Vector

(i) Generation of pGL3-AdTATA Construct (pLOR44)

An adenovirus major late promoter TATA-box motif was generated by annealing oligonucleotides to create the following with BglII and HindIII cohesive ends:

| | |
|---|---|
| BglII | HindIII |
| GATCTGGGGGGCTATAAAAGGGGGTA | (SEQ ID NO:3) |
| ACCCCCCGATATTTTCCCCCATTCGA | (SEQ ID NO:4) |

The construct was then cloned into pGL3-Basic (Promega) to generate pGL3-AdTATA.

(ii) Generation of 4×CBF1-Luc Construct (pLOR45)

A CBF-1 promoter tetramer was generated by annealing oligonucleotides to generate the following construct with XhoI and BglII cohesive ends:

| | |
|---|---|
| XhoI | BglII |
| TCGAGACCGTGGGAACTTAACCGTGGGAACTTAACCGTGGGAACTTAACCGTGGGAACTTA | (SEQ ID NO:5) |
| CTGGCACCCTTGAATTGGCACCCTTGAATTGGCACCCTTGAATTGGCACCCTTGAATCTAG | (SEQ ID NO:6) |

This construct was then cloned into pGL3-AdTATA (pLOR44: which had previously been cut with BglIIH and XhoI) to generate pLOR45.

### Example 3

### Cloning of NIC2202 and Insertion into pBluescriptSK(+), pcDNA3.1 (+) and pVP22 Vectors

PCR amplimers were constructed as follows to generate a truncated fragment of human Notch1 cDNA spanning amino acids 1759 to 2202 with a start codon (shown in bold in the NIC1759 amplimer) placed before the codon for amino acid 1759, and a stop codon placed immediately after the codon for amino acid 2202 (shown in bold in the NIC2202 amplimer). BamHI and XhoI restriction enzyme sites were also incorporated into the oligos to allow cloning of the approximately 1.3 kb fragment (shown underlined):

| | | |
|---|---|---|
| NIC1759 | 5′-AAAGGATCCACCATGGCACGCAAGCGCCGGCGCAGTCAT-3′ | (SEQ ID NO:7) |
| and | | |
| NIC2202 | 5′-GCGCCTCGAGTTAGTCCACGGGCGAGAGCAT-3′ | (SEQ ID NO:8) |

PCR was conducted using a plasmid obtained from the IMAGE Consortium (Accession Number R40977) containing a partial cDNA sequence of the human Notch1 gene as the template with the above amplimers and AdvantageII DNA polymerase (Clontech).

PCR conditions were as follows:

95° C. for 1 min −1 cycle

95° C. for 30 s, 68° C. for 3 min −30 cycles

68° C. for 3 min −1 cycle

Followed by 16° C. soak

The Notch2202 PCR fragment was cut with BamHI and XhoI and was ligated into pcDNA3.1(+) (Invitrogen) cut with BamHI and XhoI to generate pcDNA3.1-NIC2202 (pLOR34) and also into pBluescriptIISK(+) (Stratagene) cut with BamHI and AhoI to generate pBS-NIC2202 (pLOR33).

To clone the NIC2202 fragment into the Voyager Vectors pVP22/myc-His and pVP22/myc-His2 (Invitrogen), the stop codon had to be removed from the sequence to allow transcription through the myc-His tags. To remove the stop codon an oligo was constructed to build back the 3′ end of the NIC2202 sequence from a unique StuI site through to the XhoI site in the pBS-NIC2202 polylinker. A NotI site was incorporated in to the 3′ end of the oligo adjacent to the XhoI site (shown below underlined in the annealed oligos).

The annealed 5′+3′ NICStuI-XhoI oligos were as follows:

| | |
|---|---|
| StuI | |
| 5′-CCTGGCCTGTGGAAGCAAGGAGGCCAAGGACCTCAAGGCACGGAGGAAGAAGTCCC . . . | |
| 3′-GGACCGGACACCTTCGTTCCTCCGGTTCCTGGAGTTCCGTGCCTCCTTCTTCAGGG . . . | |
| NotI XhoI | |
| . . . AGGATGGCAAGGGCTGCCTGCTGGACGGCGGCCGC-3′ | (SEQ ID NO:9) |
| . . . TCCTACCGTTCCCGACGGACGACCTGCCGCCGGCGAGCT-5′ | (SEQ ID NO:10) |

As StuI is sensitive to dcm methylation, pBS-NIC2202 was transformed into a dcm⁻ strain of E. coli, SCS110 (Stratagene). pBS-NIC2202 was then cut with StuI and XhoI and the larger vector band was gel purified. The annealed 5′+3′ NICStuI-XhoI oligo was ligated into this vector, generating a modified version of pBS-NIC2202 designated pBS-NIC2202-stop. The modified version of NIC2202 without the stop codon was then cut out of this vector with BamHI and NotI and cloned into pVP22/myc-His cut with BamHI and NotI and pVP22/myc-His2 cut with BamHI and NotI yielding C-terminal and N-terminal fusions of NIC2202 with the VP22 ORF, pVP22/myc-His-NIC2202 (pLOR56) and pVP22/myc-His2-NIC2202 (pLOR57), respectively.

### Example 4

### Transfection of NIC Construct into C2C12 and Jurkat Cells and Luciferase Assay

pcDNA3.1-NIC2202 and pcDNA3.1 (as a control) were transfected with two different reporter vectors, mHES1-Luc (pLOR3) and p(4×CBF1 )-Luc (pLOR45) into both C2C12 cells and Jurkat cells (human T-cell line).

(i) C2C12 Transfections

C2C12 cells (mouse myoblast cell line) were grown in complete DMEM (DMEM plus 10% heat-inactivated FCS plus glutamine and penicillin/streptomycin), and were split regularly to prevent the cells from becoming confluent and undergoing differentiation. The day before setting up the transfections the C2C12 cells were seeded at 2.0×10⁴ cells per well of a 24-well plate in 1 ml of complete DMEM.

On the day of transfection the medium was removed from each well and replaced with 300 μl of fresh complete DMEM. Each well of C2C12 cells was transfected with 0.2 μg of reporter vector DNA (either pLOR3 or pLOR45) and 0.2 μg of either pcDNA3.1(+) or pcDNA3.1(+)-NIC2202 (pLOR34) using Effectene transfection reagent (Qiagen). All vector DNA preparations were made with Qiagen maxiprep DNA kits. The transfections were set up in quadruplicate wells of the 24-well plate. The Effectene EC buffer was added to the DNA to give a final volume of 60 μl per transfection and then 1.6 μl of Effectene Enhancer was added per transfection. The mix was vortexed for 1 second and then incubated at RT for 5 min. The tube was spun briefly and then 5 μl of Effectene Reagent was added per transfection. The mix was pipetted up and down 5 times, vortexed for 5 secs and left at RT for 10 mins to allow transfection-complex to form. The entire contents of the tube were then added to the well of a 24-well plate of C2C12 cells containing 300 μl of complete DMEM.

The transfected cells were left at 37° C. in an incubator for 24 h before doing a luciferase assay using Steady-Glo Reagent (Promega). The medium from each well was removed and replaced with 150 μl of PBS. To each well 150 μl of Steady-Glo Reagent Was then added and the plate left for 5 min at RT, before removing the entire contents of each well to white 96-well plate (Nunc) and reading the luminescence signal in a TopCount (Packard).

(ii) Jurkat Transfections

Jurkat cells (human T-cell line) [clone E6.1 (ATCC)] were grown in complete RPMI (RPMI plus 10% heat-inactivated FCS plus glutamine and penicillin/streptomycin) and were passaged the day before transfection to ensure that the cell were dividing rapidly. On the day of transfection the Jurkats were spun down and resuspended in fresh complete RPMI at 1.0×10⁶ cells/ml. Jurkats were plated out at 100 μl/well of a 96-well plate cells was transfected with 0.5 μg of reporter vector DNA (either pLOR3 or pLOR45) and 0.5 μg of either pcDNA3.1(+) or pcDNA3.1(+)-NIC2202 (pLOR34) using SuperFect transfection reagent (Qiagen). All vector DNA preparations were made with Qiagen maxiprep DNA kits. The transfections were set up in quadruplicate wells of the 96-well plate. Vector DNAs was diluted in serum-free RPMI to a final volume of 30 μl and 2 μl of SuperFect Reagent was added. The mix was pipetted up and down 5 times, vortexed for 5 secs and left at RT for 10 mins to allow transfection-complex to form. The entire contents of the tube were then added to the well of a 96-well plate of Jurkat cells.

(iii) Luciferase Assay

The transfected cells were left at 37° C. in an incubator for 24 h before doing a luciferase assay using Steady-Glo Reagent (Promega). To each well an equal volume (130 μl) of Steady-Glo Reagent was then added and the plate left for 5 min at RT, before removing the entire contents of each well to white 96-well plate (Nunc) and reading the luminescence signal in a TopCount (Packard) counter.

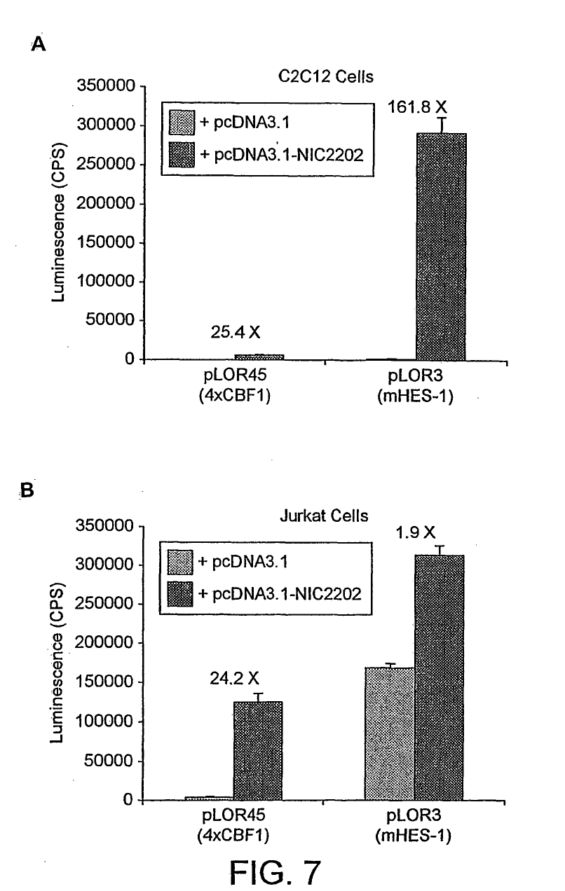

Results are shown in FIG. 7. The results show that the NIC2202 construct transactivates both reporters in both cell lines. There is a high background from the HES1-Luc vector in Jurkats and hence the fold transactivation is only 1.9×. The HES1-Luc vector was strongly transactivated in C2C12 cells.

### Example 5

### Transfection of VP22/NIC Constructs into C2C12 and Jurkat Cells and Luciferase Assay

(i) Transfection

pVP22/myc-His-NIC2202 and pVP22/myc-His2-NIC2202 were co-transfected:into C2C12 cells with the mHES1-Luc vector (pLOR3). C2C12 cells (mouse myoblast cell line) were grown in complete DMEM (DMEM plus 10% heat-inactivated FCS plus glutamine and penicillin/streptomycin), and were split regularly to prevent the cells from becoming confluent and undergoing differentiation. The day before setting up the transfections the C2C12 cells were seeded at 2.0×104 cells per well of a 24-well plate in 1 ml of complete DMEM.

On the day of transfection the medium was removed from each well and replaced with 300 μl of fresh complete DMEM. Each well of C2C12 cells was transfected with either 0.1 μg of reporter vector DNA, pmHES1-Luc(pLOR3), alone or 0.1 μg of pLOR3 and 0.2 μg of either pcDNA3.1(+)-NIC2202 (pLOR34) or 0.2 μg of pVP22/myc-His-NIC2202 (pLOR56) or 0.2 μg of pVP22/myc-His2-NIC2202 (pLOR57) using Effectene transfection reagent (Qiagen). All vector DNA preparations were made with Qiagen maxiprep DNA kits. The transfections were set up in quadruplicate wells of the 24-well plate. The Effectene EC buffer was added to the DNA to give a final volume of 60 μl per transfection and then 1.6 μl of Effectene Enhancer was added per transfection. The mix was vortexed for 1 second and then incubated at RT for 5 min. The tube was spun briefly and then 5 μl of Effectene Reagent was added per transfection. The mix was pipetted up and down 5 times, vortexed for 5 secs and left at RT for 10 mins to allow transfection-complex to form. The entire contents of the tube were then added to the well of a 24-well plate of C2C12 cells containing 300 μl of complete DMEM. An untransfected control was included in the assay.

(ii) Luciferase Assay

The transfected cells were left at 37° C. in an incubator for 24 h before doing a luciferase assay using Steady-Glo Reagent (Promega). The medium from each well was removed and replaced with 150 μl of PBS. To each well 150 μl of Steady-Glo Reagent was then added and the plate left for 5 min at RT, before removing the entire contents of each well to white 96-well plate (Nunc) and reading the luminescence signal in a TopCount (Packard).

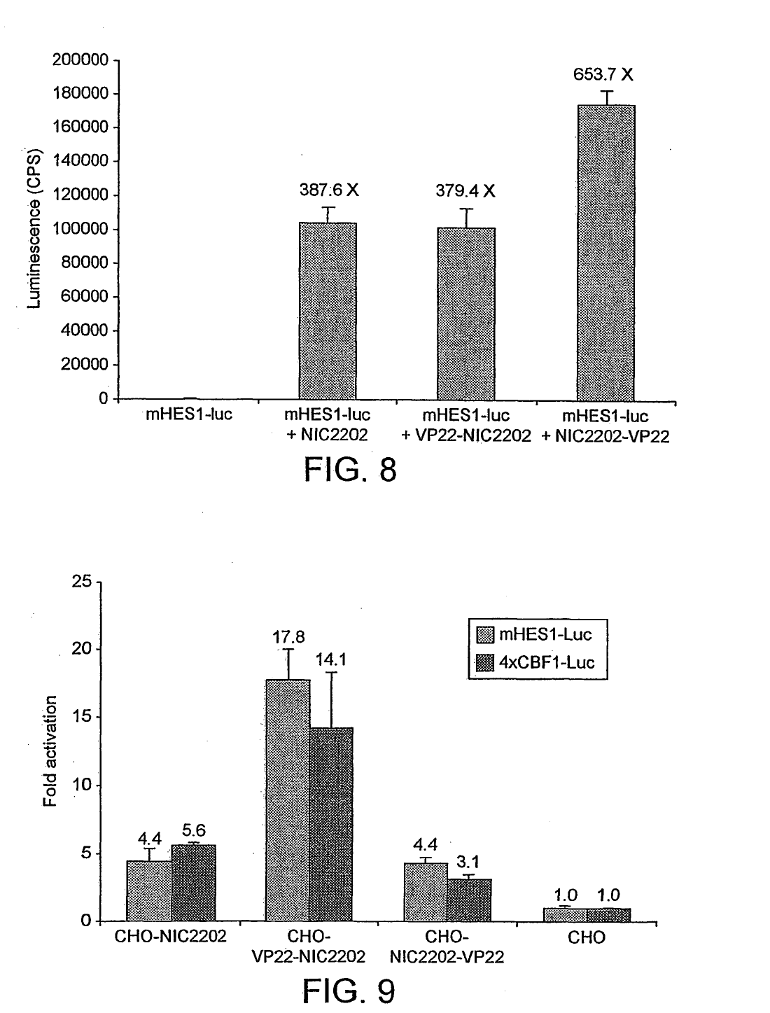

Results are shown in FIG. 8. The results show that the two VP22-NIC2202 fusion vectors also transactivate the HES1-Luc vector when co-transfected into C2C12 cells, demonstrating that the VP22-NIC2202 and NIC2202-VP22 fusion proteins are both able to transactivate a reporter vector to a similar degree to NIC2202 alone.

### Example 6

### Generation of VP22/GFP Constructs

Corresponding VP22 fusions were made with Green Fluorescent Protein (GFP) to monitor the ability of the VP22 fusion proteins to spread from cell to cell visually. PCR was used to amplify the GFP gene without a stop codon for insertion into the pVP22 vectors. The resulting PCR fragment was cut with EcoRI and NotI and cloned into pVP22/myc-His cut with EcoRI and NotI and pVP22/myc-His2 cut with EcoRI and NotI yielding C-terminal and N-terminal fusions of GFP with the VP22 ORF, pVP22/myc-His-GFP (pLOR56) and pVP22/myc-His2-GFP (pLOR57), respectively.

### Example 7

### Stable CHO Cell Transfections

CHO cells were transfected with both VP22-NIC2202 fusion vectors, pcDNA3.1-NIC2202 as a control and the two VP22-GFP fusion vectors to generate stable cell lines.

Wild-type CHO-KL cells, grown in DMEM plus 10% heat-inactivated FCS plus glutamine plus penicillin/streptomycin (P/S), were plated out at 5×10⁵ cells/well of a 6-well plate, and allowed to reach 90% confluency before transfection the following day using Lipofectamine2000 (Invitrogen). For each transfection two tubes were set up: the first with 5 μg of each maxiprep vector DNA mixed with 245 μl of OptiMem, and the second with 5 μl of Lipofectamine2000reagent in 245 μl of OptiMem. The two tubes were then combined, mixed and allowed to stand at RT for 20 min. In the meantime, the medium on each well of a 6-well plate was removed and 1.5 ml of fresh DMEM plus 10% heat-inactivated FCS plus glutamine (no P/S) added back. After the 20 min incubation of the vector DNA plus Lipofectamine2000 mix the entire contents of each transfection mix were added to a well of CHO-K1 cells containing 1.5 ml of DMEM plus 10% heat-inactivated FCS plus glutamine (no P/S). The cells were left overnight at 37° C. in a CO₂ incubator. The following day the medium was replaced with 2 ml of fresh DMEM plus 10% heat-inactivated FCS plus glutamine plus P/S and the cells left for a further 24 h at 37° C. in a C0₂ incubator.

Forty-eight hours post transfection the cells were trypsinised out of the 6-well plates, spun down, resupspended and counted. The transfected cells were then cloned by limiting dilution in complete DMEM containing 1 mg/ml G418 [Geneticin (Invitrogen)] for selection of stable transfectants. Cells were plated out at 30, 3 and 0.3 cells/well in 200 μl of complete DMEM containing 1 mg/ml G418 and left for 10-14 days to allow colonies to grow with changes of media every 3-4 days. Wells with a single growing colony of cells per well were expanded up in to a well of a 24-well plate and then into T₂₅ flasks in complete DMEM containing 0.5 mg/ml G418. Once at sufficient density in a T₂₅ flask the clones were frozen down in liquid nitrogen as well as expanded up into Tg₈₀ flasks.

Representative CHO cell clones made with pcDNA3.1-NIC2202, pVP22/myc-His-NIC2202 and pVP22/myc-His2-NIC2202 were tested by transfection with both reporter vectors, pLOR3 (MHES 1-Luc) and pLOR45 (4×CBF1-Luc). Clones tested were as follows:

| | | |
|---|---|---|
| CHO-NIC2202 | made from pcDNA3.1-NIC2202 | clone #8 |
| CHO-VP22-NIC2202 | made from pVIP22/myc-His- | clone #14 |
| | NIC2202 | |
| CHO-NIC2202-VP22 | made from pVP22/myc-His2- | clone #18 |
| | NIC2202 | |
| CHO-K1 | wild-type parent cell line control | — |

Each cell line was plated out into a 24-well plate at 1.0×10⁵ cells per well in 1 ml of complete DMEM. The following day the cells were washed with 1 ml of DMEM plus 10% heat-inactivated FCS plus glutamine (no P/S) and 0.5 ml of DMEM plus 10% heat-inactivated FCS plus glutamine (no P/S) was added back. Transfections were set up in duplicate for each reporter vector using Lipofectamine2000 (Invitrogen). For each transfection two tubes were set up: the first with 1 μg of each maxiprep reporter vector DNA mixed with OptiMem to a final volume of 50 μl, and the second with 1 μl of Lipofectamine2000 reagent in 49 μl of OptiMem. The two tubes were then combined, mixed and allowed to stand at RT for 20 min. After the 20 min incubation of the vector DNA plus Lipofectamine2000 mix the entire contents of each transfection mix were added to a well of each CHO cell clone containing 0.5 ml of DMEM plus 10% heat-inactivated FCS plus glutamine (no P/S). The transfected cells were left at 37° C. in a CO₂ incubator for 24 h before doing a luciferase assay using Steady-Glo Reagent (Promega). The medium from each well was removed and replaced with 150 μl of PBS. To each well 150 μl of Steady-Glo Reagent was then added and the plate left for 5 min at RT, before removing the entire contents of each well to white 96-well plate(Nunc) and reading the luminescence signal in a TopCount (Packard).

Results are shown in FIG. 9.

Although the levels of transactivation of the reporters were low from these stable CHO cell clones relative to high levels seen in transient co-transfection experiments they were significantly higher than the CHO cell control.

### Example 8

### Co-culture with Stable CHO Clones

C2C12 cells were transiently transfected with mHES1-Luc (pLOR3) and co-cultured with the stable CHO cells from Example 7 for 48 hours. The C2C12 cells were transfected as follows:

C2C12 cells were maintained in DMEM plus 10%heat-inactivated FCS plus glutamine plus P/S and were split regularly so as not to allow them to become over confluent which induces differentiation of the myoblasts. C2C12 cells are transfected with pLOR3 (mHES1-Luc) using Effectene transfection reagent (Qiagen) the day before addition to a 24-well plate containing the CHO cell clones. The C2C12 cells were ˜50% confluent at the time of transfection. The medium on a T₈₀ flask of C2C12 cells was replaced with 7 ml of fresh in DMEM plus 10%heat-inactivated FCS plus glutanine plus P/S. Eight μg of pLOR3 (8 μl) was mixed with 460 μl of Effectene EC buffer and then 32 μl of Effectene Enhancer was added. The tube was vortexed for 1 sec and left at RT for 5 min. Then 100 μl of Effectene Transfection Reagent was added, the tube Was mixed by vortexing for 10 sec and left at RT for 10 min. Three ml of DMEM plus 10%heat-inactivated FCS plus glutamine plus P/S was added the entire contents added to the T₈₀ flask of cells containing 7 ml of medium.

The flask of transfected C2C12 cells was left in CO₂ incubator overnight before trypsinising cells, spinning down and resuspending in 10 ml of DMEM plus 10%heat-inactivated FCS plus glutamine plus P/S. Ten μl of cells was counted and cell density adjusted to 2.0×10⁵ cells/ml with fresh DMEM plus 10%heat-inactivated FCS plus glutamine plus P/S. Transfected C2C12 cells were mixed with the CHO cell clones in a ratio of 1:1 or 5:1 of C2C12/pLOR3 cells to CHO cell clone in a 24-well plate. It was found that the ratio of 5:1 of reporter cells to CHO cell clones gave the best results so 1×10⁵ reporter cells were mixed with 2×10⁴ CHO cells in a 1 ml volume of complete DMEM and the cells were left in co-culture for 48 h in the first experiment or for 24, 48 and 72 h in the second experiment before doing a luciferase assay. The medium from each well was removed and replaced with 150 μl of PBS. To each well 150 μl of Steady-Glo Reagent was then added and the plate left for 5 min at RT, before removing the entire contents of each well to white 96-well plate (Nunc) and reading the luminescence signal in a TopCount (Packard).

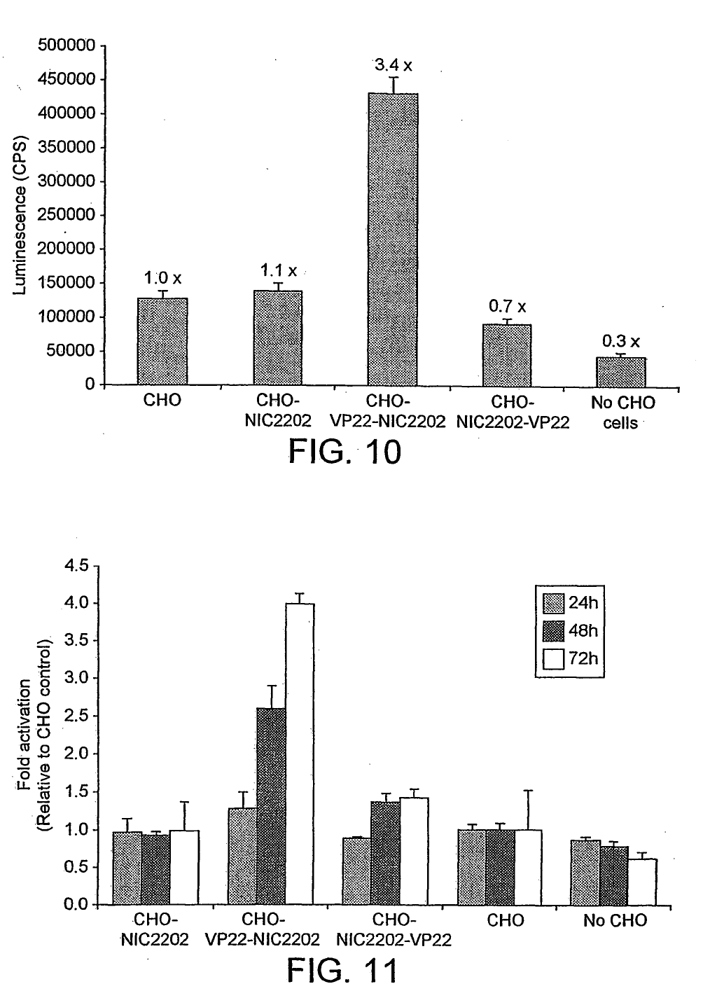

FIG. 10 shows results from two separate experiments monitoring spread in co-culture experiments.

FIG. 11 summarises the results from all these experiments with stable CHO cell clones being co-cultured with C2C12 cells. GFP-VP22 control fusion proteins (both N-terminal and C-terminal fusion) appeared to spread from CHO cell into C2C12 cells (visual determination under the fluorescence microscope).

The invention is further described by the following numbered paragraphs:

1. A conjugate comprising first and second sequences, wherein the first sequence comprises a transport protein or a polynucleotide coding for a transport protein and the second sequence comprises a polypeptide or polynucleotide for Notch signalling modulation.

2. A conjugate according to claim 1 in the form of a fusion protein.

3. A conjugate according to claim 1 or claim 2 wherein the second sequence is a polypeptide or polynucleotide for Notch signalling transduction.

4. A conjugate according to claim 3 wherein the second sequence is Notch or a fragment thereof which retains the signalling transduction ability of Notch or an analogue of Notch which has the signalling transduction ability of Notch, or a polynucleotide sequence which encodes therefor.

5. A conjugate according to claim 4 wherein the second sequence is Notch intracellular domain (Notch IC), or a polynucleotide sequence which encodes therefor.

6. A conjugate according to claim 4 wherein the second sequence is an Epstein Barr virus (EBV) protein, or a polynucleotide sequence which encodes therefor.

7. A conjugate according to claim 6 wherein the second sequence is EBNA2, BARF0 or LMP2A, or a polynucleotide sequence which encodes therefor.

8. A conjugate according to claim 1 or claim 2 wherein the second sequence is a polypeptide or polynucleotide for Notch signalling activation.

9. A conjugate according to claim 8 wherein the second sequence is a dominant negative version of a Notch signalling repressor, or a polynucleotide encoding for a dominant negative version of a Notch signalling repressor.

10. A conjugate according to claim 8 wherein the second sequence is a polypeptide or polynucleotide which inhibits the expression or activity of a Notch signalling repressor, or a polynucleotide encoding for such a polypeptide.

11. A conjugate according to claim 1 or claim 2 wherein the second sequence is an agent which acts in the nucleus or a polynucleotide which codes for such an agent.

12. A conjugate according to claim 1 or claim 2 wherein the second sequence is a Notch signalling transcription factor or a polynucleotide which codes for a Notch signalling transcription factor.

13. A conjugate according to claim 1 or claim 2 wherein the second sequence is a DNA binding agent or a polynucleotide which codes for a DNA binding agent.

14. A conjugate according to any one of the preceding claims wherein the second sequence comprises a Notch Anlrin domain or a polynucleotide which codes for a Notch Ankyrin domain.

15. A conjugate according to claim 14 wherein the second sequence further comprises a RAM domain, a PEST sequence or an OPA sequence or a polynucleotide which codes for such a sequence.

16. A conjugate according to claim 1 or claim 2 wherein the second sequence is a polypeptide or polynucleotide for Notch signalling inhibition.

17. A conjugate according to claim 16 wherein the second sequence is a dominant negative version of a Notch signalling activator or transducer, or a polynucleotide encoding for a dominant negative version of a Notch signalling activator or transducer.

18. A conjugate according to claim 16 wherein the second sequence is a polypeptide or polynucleotide which inhibits the expression or activity of a Notch signalling activator or transducer, or a polynucleotide encoding for such a polypeptide.

19. A conjugate according to claim 16 wherein the second sequence is a polypeptide capable of downregulating the expression or activity of Notch, a Notch ligand or a downstream component of the Notch signalling pathway, or a polynucleotide which encodes therefor.

20. A conjugate according to claim 19 wherein the second sequence is selected from Toll-like receptors, bone morphogenic proteins (BMPs), BMP receptors, activins and derivatives, fragments, variants and homologues thereof, or a polynucleotide which encodes therefor.

21. A conjugate according to any preceding claim wherein the first sequence is a nuclear localisation protein.

22. A conjugate according to any preceding claim wherein the first sequence is a herpesvirus VP22 protein (VP22) or a fragment thereof that retains a VP22 transport function.

23. A conjugate according to claim 22 wherein the first sequence is a full length VP22 sequence.

24. A conjugate according to claim 22 wherein the fragment of VP22 comprises: from about amino acid 60 to about amino acid 301 of the full length VP22 sequence, or from about amino acid 159 to about amino acid 301 of the full length. VP22 sequence.

25. A conjugate according to any one of claims 1 to 20 wherein the first sequence comprises a homeodomain, or a variant thereof that retains a transport function.

26. A conjugate according to claim 25 wherein the homeodomain is from Antennapedia, Fushi-tarazu or Engrailed.

27. A conjugate according to any one of claims 1 to 20 wherein the first sequence is an HIV tat protein, or a variant thereof that retains a transport function.

28. A polynucleotide sequence encoding the conjugate of any preceding claim.

29. An expression vector comprising the polynucleotide sequence of claim 28.

30. A host cell transformed with the expression vector of claim 29.

31. A method for preparing a conjugate comprising culturing the host cell of claim 30 under conditions which provide for the expression of the conjugate.

32. A conjugate prepared by the method of claim 31.

33. A method of transporting a protein for, Notch signalling modulation or a polynucleotide sequence which encodes therefor into a cell comprising exposing a cell to a fusion protein according to any one of claims 1 to 27 or 32.

34. A pharmaceutical composition comprising the conjugate of any of claims 1 to 27 or 32 and a pharmaceutically acceptable excipient, diluent or carrier.

35. A pharmaceutical composition according to claim 34 for use in the treatment of T-cell mediated disease.

36. Use of the conjugate of any of claims 1 to 27 or 32 in the preparation of a medicament for the prevention and/or treatment of disease or infection.

37. Use according to claim 36 wherein the disease is a T-cell mediated disease.

38. A conjugate, polynucleotide sequence, expression vector, host cell, method, pharmaceutical composition or use substantially as hereinbefore described with reference to the accompanying Figures.

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are apparent to those skilled in molecular biology or related fields are intended to be within the scope of the following claims.

### References (incorporated herein by reference thereto)

Elliott &. O'Hare (1997) Cell 88:223-233.

Hsieh et al. (1996) Molecular & Cell Biology 16(3):952-959.

Strobl et al. (2000) J Virol 74(4):1727-35.

Kusano and Raab-Truab (2001) J Virol 75(1):384-395.

Jeon et al. (2000) Mol Cells 10(6):728-32.

Gehring et al. (1990) Trends Genet 6:323-9.

Derossi D et al. (1994) J Biol Chem 269:10444-10450.

Derossi D et al. (1996) J Biol Chem 271:18188-18193.

Joliot AH et al. (1991) The New Biol 3:1121-1134 and PNAS (1991) 88:1864-1868.

Perez F et al. (1992) J Cell Sci 102:712-722.

Brugidou J et al. (1995) Biochem Biophys Res Comm 214(2):685-693.

Frankel A. D. and Pabo C. O. (1988) Cell 55:1189-93.

Ruben, S. et al. (1989) J. Virol, 63:1-8.

Hauber, J. et al. (1989) J. Virol, 63:1181-1187.

Frankel, A. D. et al. (1988) Science 240:70-73

Frankel, A. D. et al. (1988) Proc. Natl. Acad. Sci USA 85:6297-6300.

Garcia, J. A. et al. (1988) EMBO J. 7:3143.

Sadaie, M. R. et al. (1989) J. Virol 63:1.

Bachmair, A. et al. (1989) Cell 56:1019-1032.

Lu, F. M. et al. (1996) Proc Natl Acad Sci 93(11):5663-7.

Lieber, T. et al. (1993) Genes Dev 7(10):1949-65.

Struhi, G. et al. (1998) Cell 93(4 :649-60.

Matsuno, K. et al. (1995) Development 121(8):2633-44.

Schroeter, E.H. et al. (1998) Nature 393(6683):382-6.

Jarriault S. et al. (1995) Nature 377(6547):355-8.

Takebayashi K. et al. (1994) J Biol Chem 269(7):150-6.

Leimeister C. et a!. (1999) Mech Dev 85(1-2):173-7.

Chee M. et al. (1996) Science 274:601-614.

Camilli et al. (1994) Proc Natl Acad Sci USA 91:2634-2638.

Pasqualini R, Ruoslahti E. (1996) Nature 380:364-366.

Ruoslahti (1996) E. Ann. Rev. Cell Dev. Biol. 12:697-715.

Arap, W, Pasqualini, R, Ruoslahti, E (1998) Science 279:377-380.

Ordentlich et al. (1998) Mol. Cell. Biol. 18:2230-2239.

Tamura K, et al. (1995) Curr. Biol. 5:1416-1423.

Artavanis-Tsakonas S, et al. (1995) Science 268:225-232.

Munro S, Freeman M. (2000) Curr. Biol. 10:813-820.

Ju BJ, et al. (2000) Nature 405:191-195.

Moloney DJ, et al. (2000) Nature 406:369-375.

Brucker K, et al. (2000) Nature 406:411-415.

Panin VM, et al. (1997) Nature 387:908-912.

Hicks C, et al. (2000) Nat. Cell. Biol. 2:515-520.

Irvine KD (1999) Curr. Opin. Genet. Dcvel. 9:434-441.

Schroeter EH, et al. (1998) Nature 393:382-386.

Struhi G, Adachi A. (1998) Cell 93:649-660.

Weinmaster G. (2000) Curr. Opin. Genet. Dev. 10:363-369.

Artavanis-Tsakonas S,. et al. (1999) Science 284:770-776.

Osborne B, Miele L. (1999) Immunity 11:653-663.

Matsuno K, et al. (1998) Nat. Genet. 19:74-78.

Medhzhitov et al. (1997) Nature 388:394-397.

Li et al. (1998) Immunity 8(1):43-55.

Iemura et al. (1998) PNAS 95:9337-9345.

Valenzuela et al. (1995) J. Neurosci. 15:6077-6084.

Sasai et al. (1994) Cell 79:779-790.

Wilson and Hemmati-Brivanlou (1997) Neuron 18:699-710.

Hemmati-Brivanlou and Melton (1997) Cell 88:13-17.

Dkuz et al. (1997) Cell 90: 271-280.

Zhao et al. (1995) J. hmmunol. 155:3904-3911.

Hoyne et al. (2000)-reference in press.

We claim:

1. A conjugate comprising a first sequence and a second sequence, wherein the first sequence comprises a transport protein or a polynucleotide encoding a transport protein and the second sequence comprises a polypeptide or polynucleotide that modulates Notch signalling.

2. The conjugate according to claim 1, wherein the conjugate is a fusion protein.

3. The conjugate according to claim 1, wherein the second sequence. is a polypeptide or polynucleotide for Notch signalling transduction.

4. The conjugate according to claim 3, wherein the second sequence is Notch, or a fragment thereof which retains the signalling transduction ability of Notch, or an analogue of Notch which has the signalling transduction ability of Notch, or a polynucleotide sequence which encodes therefor.

5. The conjugate according to claim 4, wherein the second sequence is Notch intracellular domain (Notch IC), or a polynucleotide sequence that encodes therefor.

6. The conjugate according to claim 4, wherein the second sequence is an Epstein Barr virus (EBV) protein, or a polynucleotide sequence that encodes therefor.

7. The conjugate according to claim 6, wherein the second sequence is EBNA2, BARF0 or LMP2A, or a polynucleotide sequence that encodes therefor.

8. The conjugate according to claim 1, wherein the second sequence is a polypeptide or polynucleotide that activates Notch signalling.

9. The conjugate according to claim 8, wherein the second sequence is a dominant negative version of a Notch signalling repressor, or a polynucleotide encoding a dominant negative version of a Notch signalling repressor.

10. The conjugate according to claim 8, wherein the second sequence is a polypeptide or polynucleotide which inhibits the expression or activity of a Notch signalling repressor, or a polynucleotide encoding said polypeptide.

11. The conjugate according to claim 1, wherein the second sequence is an agent that acts in the nucleus or a polynucleotide encoding said agent.

12. The conjugate according to claim 1, wherein the second sequence is a Notch signalling transcription factor or a polynucleotide encoding a Notch signalling transcription factor.

13. The conjugate according to claim 1, wherein the second sequence is a DNA binding agent or a polynucleotide encoding a DNA binding agent.

14. The conjugate according to claim 1, wherein the second sequence is a polypeptide comprising a Notch Ankrin domain or a polynucleotide encoding a polypeptide comprising a Notch Ankyrin domain.

15. The conjugate according to claim 14, wherein the second sequence further comprises a RAM domain, a PEST sequence, an OPA sequence or a polynucleotide encoding a RAM domain, a PEST sequence or an OPA sequence.

16. The conjugate according to claim 1, wherein the second sequence is a polypeptide or polynucleotide that inhibits Notch signalling.

17. The conjugate according to claim 16, wherein the second sequence is a dominant negative version of a Notch signalling activator or transducer, or a polynucleotide encoding a dominant negative version of a Notch signalling activator or transducer.

18. The conjugate according to claim 16, wherein the second sequence is a polypeptide or polynucleotide that inhibits the expression or activity of a Notch signalling activator or transducer, or a polynucleotide encoding a polypeptide that inhibits the expression or activity of a Notch signalling activator or transducer.

19. The conjugate according to claim 16, wherein the second sequence is a polypeptide capable of downregulating the expression or activity of Notch, a Notch ligand or a downstream component of the Notch signalling pathway, or a polynucleotide that encodes therefor.

20. The conjugate according to claim 19, wherein the second sequence is selected from the group consisting of Toll-like receptors, bone morphogenic proteins (BMPs), BMP receptors, activins, derivatives, fragments, variants and homologues thereof, and a polynucleotide that encodes therefor.

21. The conjugate according to claim 1, wherein the first sequence is a nuclear localisation protein.

22. The conjugate according to claim 1, wherein the first sequence is a herpesvirus VP22 protein (VP22) or a fragment thereof that retains a VP22 transport function.

23. The conjugate according to claim 22, wherein the first sequence is a full length VP22 sequence.

24. The conjugate according to claim 22, wherein the fragment of VP22 comprises:
from about amino acid 60 to about amino acid 301 of the full length VP22 sequence, or
from about amino acid 159 to about amino acid 301 of the full length VP22 sequence.

25. The conjugate according to claim 1, wherein the first sequence comprises a homeodomain, or a variant thereof that retains a transport function.

26. The conjugate according to claim 25, wherein the homeodomain is from Antennapedia, Fushi-tarazu or Engrailed.

27. The conjugate according to claim 1, wherein the first sequence is an HIV tat protein, or a variant thereof that retains a transport function.

28. A polynucleotide sequence encoding the conjugate of claim 1.

29. An expression vector comprising the polynucleotide sequence of claim 28.

30. A host cell transformed with the expression vector of claim 29.

31. A method for preparing a conjugate comprising culturing the host cell of claim 30 under conditions which provide for the expression of the conjugate.

32. A conjugate prepared by the method of claim 31.

33. A method of transforming a cell with a protein for Notch signalling modulation or a polynucleotide sequence which encodes therefor, the method comprising introducing the expression vector of claim 29 into the cell.

34. A composition comprising the conjugate of claim 1 and a pharmaceutically acceptable excipient, diluent or carrier.

35. A method for the prevention or treatment of disease or infection comprising administering the composition of claim 34 to a subject in need thereof.

36. The method according to claim 35, wherein the disease is a T-cell mediated disease.
